(19) United States
(12) Patent Application Publication
(10) Pub. No.: US 2014/0179875 A1
(43) Pub. Date: Jun. 26, 2014

(54) SULFURIZATION REAGENTS ON SOLID SUPPORTS

(71) Applicants: Wieslaw Adam Mazur, Mason, OH (US); Victor Sorokin, Cincinnati, OH (US); Steven Karl Laughlin, Independence, KY (US)

(72) Inventors: Wieslaw Adam Mazur, Mason, OH (US); Victor Sorokin, Cincinnati, OH (US); Steven Karl Laughlin, Independence, KY (US)

(73) Assignee: GIRINDUS AMERICA, INC., Cincinnati, OH (US)

(21) Appl. No.: 14/235,852

(22) PCT Filed: Jul. 24, 2012
(86) PCT No.: PCT/EP2012/064455

§ 371 Date: Jan. 29, 2014

Related U.S. Application Data
(60) Provisional application No. 61/513,076, filed on Jul. 29, 2011.

(51) Int. Cl. C08F 112/08; C07H 21/04

(52) U.S. Cl. CPC C08F 112/08; C07H 21/04
USPC 525/333.5; 536/25.31

(57) ABSTRACT

Described herein are novel solid-supported sulfurization reagents having the general structure: (I) wherein (P) is a polymer; X is a linker; R₁ is an alkyl group, a cycloalkyl group, an aryl group, or a heterocycle; and R₂ is an alkyl group, an aryl group, a methyleneacyloxy group having the formula —CH₂—O—C(O)—R₇, a methylene carbonate group having the formula —CH₂—O—C(O)—OR₈, or a methylene carbamate group having the formula —CH₂—O—C(O)—NR₉R₁₀, wherein R₇ is a C₁ to C₂₀ hydrocarbon residue, R₈ is any alkyl, cycloalkyl, aryl, or heteroaryl, and R₉ and R¹⁰ are independently hydrogen, alkyl, cycloalkyl, aryl, or heteroaryl. Other embodiments include solid-supported sulfurization reagents having the structure of Formula I, wherein (P) is a polystyrene-based solid support and X is an aromatic linker. Also described herein are methods for synthesizing the solid-supported sulfurization reagents and their use during the synthesis of oligonucleotides.

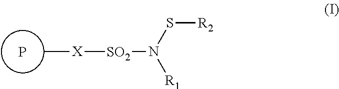

### CROSS-REFERENCE TO RELATED APPLICATIONS

This application claims priority to U.S. provisional application No. 61/513,076 filed on 29 Jul. 2011, the whole content of this application being incorporated herein by reference for all purposes.

### TECHNICAL FIELD OF THE INVENTION

Described herein are novel solid-supported sulfurization reagents useful for the preparation of phosphorothioate oligonucleotides using the H-phosphonate method. Also described herein are methods for synthesizing these novel solid-supported sulfurization reagents and methods of their use to prepare phosphorothioate oligonucleotides using the H-phosphonate method.

### BACKGROUND OF THE INVENTION

Oligonucleotides belong to a class of biopharmaceuticals with great potential for therapies of various diseases including, for example, cancer, viral infections, and inflammatory disease. An important approach to advancing oligonucleotides as therapeutics involve modifications of the oligomer backbone to provide, among other things, metabolic resistance, chemical stability and improve in vivo transport to the site of action. Examples of modified backbone chemistries include, without limitation: peptide nucleic acids (PNAs) (see Nielsen, Methods Mol. Biol., 208:3-26, 2002), locked nucleic acids (LNAs) (see Petersen & Wengel, Trends Biotechnol., 21(2):74-81, 2003), phosphorothioates (see Eckstein, Antisense Nucleic Acid Drug Dev., 10(2):117-21, 2000), methylphosphonates (see Thiviyanathan et al., Biochemistry, 41(3):827-38, 2002), phosphoramidates (see Gryaznov, Biochem. Biophys. Acta, 1489(1): 131-40, 1999; Pruzan et al., Nucleic Acids Res., 30(2):559-68, 2002), thiophosphoramidates (see, e.g., Gryaznov, et al., Nucleosides Nucleotides Nucleic Acids, 20(4-7):401-10, 2001; Herbert, et al., Oncogene, 21(4):638-42, 2002). Formation of phosphorothioates belongs to one of the most useful modifications since the replacement of the P═O moiety with a P═S moiety makes the oligonucleotides resistant to nucleolytic degradation while retaining in most cases the biological properties of natural oligomers.

Phosphorothioates can be formed by oxidative sulfurization (Oligonucleotide synthesis, methods and applications, P. Herdewijn Methods in Molecular Biology, volume 288, Chapter 4, 51-63). There are basically two approaches to making phosphorothioates, both of which depend upon the nature of phosphorous esters used for this reaction and the expected products. First, an unsubstituted sulfur atom may be introduced to the phosphorus atom by means of, for example, elemental sulfur, dibenzoyl tetrasulfide, 3-H-1,2-benzodithiol-3-one 1,1-dioxide (also known as Beaucage reagent, (Iyer, et al., J. Org. Chem. 55, 4693-4699 (1990)), tetraethylthiuram disulfide (TETD), dimethylthiuram disulfide (DTD), phenylacetyl disulfide (PADS), and bis(O,O-diisopropoxy phosphinothioyl) disulfide (also known as Stec's reagent). These reactions are often used in the automated synthesis of oligonucleotides attached to solid supports by the phosphoramidite method and comprise the oxidative sulfurization of phosphorus triesters formed during the elongation reaction of oligomers

Second, oligomeric phosphorothioates may be foamed using the H-phosphonate method, which involves the reaction of a H-phosphonate diester with a sulfur transfer reagent, wherein the sulfur atom, bearing an aliphatic or aromatic substituent, is transferred to phosphorus. The aliphatic or aromatic substituent at sulfur serves as a protecting group during the synthetic operation and usually is cleaved at the final stage of oligonucleotide preparation to yield the oligomeric phosphorthioates. This method is often used for the synthesis of oligonucleotides in solution.

In contrast to the relatively large selection of reagents available for introducing the unsubstitued sulfur atom to phosphorus esters using the phosphoramidite method, the spectrum of sulfurization reagents suitable for sulfurization of H-phosphonate esters is limited (see, e.g. Dreef, et al. Synlett, 481-483, 1990; U.S. Pat. No. 6,506,894). Practically, only the cyanoethylsulfide group has been commercially used in this reaction during the solution synthesis of oligonucleotides, requiring chromatographic purification at each step. A critical problem in the solution synthesis of oligonucleotides concerns the necessity to obtain high substrate conversions with excellent specificity at each synthetic step giving high purity products in a form that facilitates simple purification, in particular avoiding chromatography. Given the lack of methods allowing for economical solution phase synthesis, the solution phase technology does not seem to be currently used for commercial scale oligonucleotide synthesis.

Further, known solution phase sulfurization reagents are generally small molecules, which are used as soluble agents during sulfurization of oligonucleotides in solution using the H-phosphonate method. Accordingly, these sulfurization reagents are known to yield undesirable by-products in the sulfurization reaction medium, which are then typically removed from the reaction medium either by extraction, solvent-assisted precipitation, or chromatography. These purification techniques are costly, time consuming, and generate substantial solvent waste. Moreover, the synthesis of these sulfurization reagents typically requires costly and time-consuming purification techniques. The solid-supported sulfurization reagents described herein ameliorate these problems. They exhibit desired reactivity as solution phase sulfurization reagents in oligonucleotide synthesis using the H-phosphonate method, provide an efficient method of purifying the sulfurized oligonucleotide by filtration, and provide an efficient method of synthesizing sulfurization reagents purified by filtration.

### SUMMARY OF THE INVENTION

Described herein are novel solid-supported sulfurization reagents having the structure:

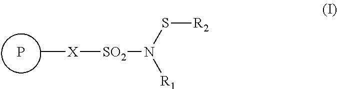

wherein (P) is a polymer; X is a linker; R₁ is an alkyl group, a cycloalkyl group, an aryl group, or a heterocycle; and R₂ is an alkyl group, an aryl group, a methyleneacyloxy group having the formula —CH₂—O—C(O)—R₇, a methylene carbonate group having the formula —CH₂—O—C(O)—OR₈, or a methylene carbamate group having the formula —CH₂—O—C(O)—NR₉R₁₀, wherein R₇ is a C₁ to C₂₀ hydrocarbon residue, R₈ is any alkyl, cycloalkyl, aryl, or heteroaryl, and R₉ and R₁₀ are independently hydrogen, alkyl, cycloalkyl, aryl, or heteroaryl. Other embodiments include solid-supported sulfurization reagents having the structure of Formula I, wherein (P) is a polystyrene-based solid support and X is an aromatic linker.

Also described herein are methods for synthesizing the solid-supported sulfurization reagents and their use during the synthesis of oligonucleotides.

Other embodiments, objects, features and advantages will be set forth in the detailed description of the embodiments that follows, and in part will be apparent from the description or may be learned by practice of the claimed invention. These objects and advantages will be realized and attained by the processes and compositions described and claimed herein. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### DETAILED DESCRIPTION OF THE PREFERRED EMBODIMENTS

While the present disclosure is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading. Embodiments disclosed herein are inclusive and exclusive of other embodiments.

Sulfurization Reagents

As used herein, the term “aryl group” denotes an aromatic carbocyclic ring system, wherein the ring may comprise from 3 annular carbon atoms to 24 annular carbon atoms. In one embodiment, the ring may comprise 3 annular carbon atoms, 4 annular carbon atoms, 5 annular carbon atoms, 6 annular carbon atoms, 7 annular carbon atoms, 8 annular carbon atoms, 9 annular carbon atoms, 10 annular carbon atoms, 11 annular carbon atoms, 12 annular carbon atoms, 13 annular carbon atoms, 14 annular carbon atoms, 15 annular carbon atoms, 16 annular carbon atoms, 17 annular carbon atoms, 18 annular carbon atoms, 19 annular carbon atoms, 20 annular carbon atoms, 21 annular carbon atoms, 22 annular carbon atoms, 23 annular carbon atoms, or 24 annular carbon atoms. The aryl group may be unsubstituted or independently substituted, for example, with one or more aryl or heteroaryl groups, alkyl groups, cycloalkyl groups, or functional groups. In one embodiment, the aryl group may be ortho-substituted, meta-substituted, or para-substituted with one or more alkyl groups, halogens, or aryl groups.

As used herein, the term “heteroatom” denotes any atom that is not carbon or hydrogen. By way of example and without limitation, suitable heteroatoms include boron, aluminum, gallium, indium, thallium, silicon, germanium, tin, lead, nitrogen, phosphorus, arsenic, antimony, bismuth, oxygen, sulfur, selenium, tellurium, polonium, fluorine, chlorine, bromine, iodine, and astatine.

As used herein, the term “heteroaryl group” denotes an aromatic carbocyclic system, wherein the ring may comprise from 3 annular atoms to 24 annular atoms, at least one of which is any heteroatom. In one embodiment, the ring may comprise 3 annular atoms, 4 annular atoms, 5 annular atoms, 6 annular atoms, 7 annular atoms, 8 annular atoms, 9 annular atoms, 10 annular atoms, 11 annular atoms, 12 annular atoms, 13 annular atoms, 14 annular atoms, 15 annular atoms, 16 annular atoms, 17 annular atoms, 18 annular atoms, 19 annular atoms, 20 annular atoms, 21 annular atoms, 22 annular atoms, 23 annular atoms, or 24 annular atoms, at least one of which is a heteroatom. In one embodiment, the ring may comprise 1 annular heteroatom, 2 independent or the same annular heteroatoms, 3 independent or the same annular heteroatoms, 4 independent or the same annular heteroatoms, 5 independent or the same annular heteroatoms, 6 independent or the same annular heteroatoms, 7 independent or the same annular heteroatoms, or 8 independent or the same annular heteroatoms. Suitable heteroatoms include, but are not limited to, boron, aluminum, gallium, indium, thallium, silicon, germanium, tin, lead, nitrogen, phosphorus, arsenic, antimony, bismuth, oxygen, sulfur, selenium, tellurium, polonium, fluorine, chlorine, bromine, iodine, and astatine. In one embodiment, the heteroaryl group may independently comprise one or more annular nitrogen atoms, annular oxygen atoms, or annular sulfur atoms.

As used herein, the term “alkyl group” denotes any linear, secondary branched, or tertiary branched alkyl substituent. In one embodiment, the linear, secondary branched, or tertiary branched alkyl substituent may comprise from 1 carbon atom to 20 carbon atoms. In another embodiment, the linear, secondary branched, or tertiary branched alkyl substituent may comprise 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, or 20 carbon atoms.

By way of example and without limitation, suitable alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)CH₂CH₂CH₃), 1-heptyl (—CH₂CH₂CH₂CH₂CH₂CH₂CH₃), 2-heptyl (—CH(CH₃)CH₂CH₂CH₂CH₂CH₃), 3-heptyl (—CH(CH₂CH₃)CH₂CH₂CH₂CH₃), 4-heptyl (—CH(CH₂CH₂CH₃)₂), 1-octyl (—CH₂CH₂CH₂CH₂CH₂CH₂CH₂CH₃), 2-octly (—CH(CH₃)CH₂CH₂CH₂CH₂CH₂CH₃), 3-octyl ((—CH(CH₂CH₃)CH₂CH₂CH₂CH₂CH₃), 4-octyl (—CH(CH₂CH₂CH₃)CH₂CH₂CH₂CH₃), and the like.

As used herein, the term “cycloalkyl group” denotes a non-aromatic monocyclic or multicyclic ring system, wherein the ring comprises from 3 annular carbon atoms to 10 annular carbon atoms. In one embodiment, the ring comprises 3 annular carbon atoms, 4 annular carbon atoms, 5 annular carbon atoms, 6 annular carbon atoms, 7 annular carbon atoms, 8 annular carbon atoms, 9 annular carbon atoms, or 10 annular carbon atoms. By way of example and without limitation, suitable monocyclic cycloalkyl groups include cyclopropyl, cylcobutyl, cyclopentyl, cyclohexyl, cycloheptyl, cyclooctyl, and cyclononyl groups. Exemplary multicyclic cycloalkyl groups include perhydronaphthyl, adamant-(1- or 2-)yl and norbornyl and spirocyclic groups such as spiro[2,2]pentane, spiro[2,5]octane, spiro[4,4]non-2yl, and the like.

As used herein, the term “functional group” denotes a substituent comprising any heteroatom. In one embodiment, the heteroatom may be boron, aluminum, gallium, indium, thallium, silicon, germanium, tin, lead, nitrogen, phosphorus, arsenic, antimony, bismuth, oxygen, sulfur, selenium, tellurium, polonium, fluorine, chlorine, bromine, iodine, and astatine. A functional group may comprise any desired number of heteroatoms. In one embodiment, a functional group may contain 1 heteroatom, 2 heteroatoms, 3 heteroatoms, 4 heteroatoms, 5 heteroatoms, or 6 heteroatoms. A functional group may also comprise any desired number of atoms. In one embodiment, a functional group may contain 1 atom, 2 atoms, 3 atoms, 4 atoms, 5 atoms, 6 atoms, 7 atoms, 8 atoms, 9 atoms, 10 atoms, 11 atoms, 12 atoms, 13 atoms, 14 atoms, 15 atoms, 16 atoms, 17 atoms, 18 atoms, 19 atoms, 20 atoms, 21 atoms, 22 atoms, 23 atoms, 24 atoms, 25 atoms, 26 atoms, 27 atoms, 28 atoms, 29 atoms, 30 atoms, 31 atoms, 32 atoms, 33 atoms, 34 atoms, 35 atoms, 36 atoms, 37 atoms, 38 atoms, 39 atoms, 40 atoms, 41 atoms, 42 atoms, 43 atoms, 44 atoms, 45 atoms, 46 atoms, 47 atoms, 48 atoms, 49 atoms, or 50 atoms. By was of example and without limitation, suitable functional groups include halogens, a hydroxyl group, an alkoxy group, an aryloxy group, a mercapto group, an amino group, a nitro group, a carbonyl group, an acyl group, an optionally esterified carboxyl group, a carboxamide group, a urea group, a urethane group, and the thiol derivatives of the aforementioned groups containing a carbonyl group, a phosphine group, a phosphonate group, a phosphate group, a sulfoxide group, a sulfone group, and a sulfonate group.

The term “hydrocarbon residue” denotes any group containing at least one C—H moiety. In one embodiment, the hydrocarbon residue may by a linear or branched alkyl or alkylene group, which may optionally contain one or more independent heteroatoms such as boron, aluminum, gallium, indium, thallium, silicon, germanium, tin, lead, nitrogen, phosphorus, arsenic, antimony, bismuth, oxygen, sulfur, selenium, tellurium, polonium, fluorine, chlorine, bromine, iodine, and astatine. The linear or branched alkyl or alkylene group may optionally contain one or more independent cycyloalkyl groups, heterocycles, aromatic systems, and functional groups. The hydrocarbon residue may comprise one or more independent multiple bounds, including both conjugated and unconjugated double or triple bonds. In another embodiment, the hydrocarbon residue may be aromatic or heteroaromatic.

The hydrocarbon residue may comprise at least 1 carbon atom. In one embodiment, the organic residue comprises 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, 20 carbon atoms, 21 carbon atoms, 22 carbon atoms, 23 carbon atoms, 24 carbon atoms, 25 carbon atoms, 26 carbon atoms, 27 carbon atoms, 28 carbon atoms, 29 carbon atoms, 30 carbon atoms, 31 carbon atoms, 32 carbon atoms, 33 carbon atoms, 34 carbon atoms, 35 carbon atoms, 36 carbon atoms, 37 carbon atoms, 38 carbon atoms, 39 carbon atoms, 40 carbon atoms, 41 carbon atoms, 42 carbon atoms, 43 carbon atoms, 44 carbon atoms, 45 carbon atoms, 46 carbon atoms, 47 carbon atoms, 48 carbon atoms, 49 carbon atoms, 50 carbon atoms, 51 carbon atoms, 52 carbon atoms, 53 carbon atoms, 54 carbon atoms, 55 carbon atoms, 56 carbon atoms, 57 carbon atoms, 58 carbon atoms, 59 carbon atoms, 60 carbon atoms, 61 carbon atoms, 62 carbon atoms, 63 carbon atoms, 64 carbon atoms, 65 carbon atoms, 66 carbon atoms, 67 carbon atoms, 68 carbon atoms, 69 carbon atoms, 70 carbon atoms, 71 carbon atoms, 72 carbon atoms, 73 carbon atoms, 74 carbon atoms, 75 carbon atoms, 76 carbon atoms, 77 carbon atoms, 78 carbon atoms, 79 carbon atoms, 80 carbon atoms, 81 carbon atoms, 82 carbon atoms, 83 carbon atoms, 84 carbon atoms, 85 carbon atoms, 86 carbon atoms, 87 carbon atoms, 88 carbon atoms, 89 carbon atoms, 90 carbon atoms, 91 carbon atoms, 92 carbon atoms, 93 carbon atoms, 94 carbon atoms, 95 carbon atoms, 96 carbon atoms, 97 carbon atoms, 98 carbon atoms, 99 carbon atoms, or 100 carbon atoms.

When the hydrocarbon residue contains one or optionally more double bonds, it may comprise an alkenyl or cycloalkenyl group containing from 1 carbon atom to 20 carbon atoms. In one embodiment, the alkenyl or cycloalkenyl group may comprise 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, or 20 carbon atoms. Exemplary alkenyl and cycloalkenyl groups include, without limitation, vinyl, 1-allyl, 2-allyl, n-but-2-enyl, isobutenyl, 1,3-butadienyl, cyclopentenyl, and styryl.

When the hydrocarbon residue contains one or optionally more triple bonds, it may comprise an alkynyl group containing from 1 carbon atom to 20 carbon atoms. In one embodiment, the alkynyl group may comprise 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, or 20 carbon atoms. Exemplary alkynyl groups include, without limitation, 1-propynyl, 2-propynyl, n-but-2-ynyl, and 2-phenylethynyl.

When the hydrocarbon residue contains one or optionally more aromatic systems, it may comprise an aryl, alkylaryl, or aromatic heterocyclic group having from 3 carbon atoms to 24 carbon atoms. In one embodiment, the aryl, alkylaryl, or aromatic heterocyclic group may comprise 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, 20 carbon atoms, 21 carbon atoms, 22 carbon atoms, 23 carbon atoms, or 24 carbon atoms. Exemplary aromatic systems include, without limitation, phenyl, 1-tolyl, 2-tolyl, 3-tolyl, xylyl, 1-naphthyl, and 2-naphthyl.

As used herein, the term “alkylene group” or “cycloalkylene group” denotes the divalent radicals derived from the alkyl or cycloalkyl groups as defined above.

As used herein, the term “heterocycle” denotes a cyclic system comprising at least one saturated or unsaturated ring, wherein the cyclic system is made up of 3 annular atoms, 4 annular atoms, 5 annular atoms, 6 annular atoms, 7 annular atoms, 8 annular atoms, 9 annular atoms, 10 annular atoms, 11 annular atoms, 12 annular atoms, 13 annular atoms, 14 annular atoms, 15 annular atoms, 16 annular atoms, 17 annular atoms, 18 annular atoms, 19 annular atoms, 20 annular atoms, 21 annular atoms, 22 annular atoms, 23 annular atoms, 24 annular atoms, 25 annular atoms, 26 annular atoms, 27 annular atoms, 28 annular atoms, 29 annular atoms, 30 annular atoms, 31 annular atoms, 32 annular atoms, 33 annular atoms, 34 annular atoms, 35 annular atoms, 36 annular atoms, 37 annular atoms, 38 annular atoms, 39 annular atoms, 40 annular atoms, 41 annular atoms, 42 annular atoms, 43 annular atoms, 44 annular atoms, 45 annular atoms, 46 annular atoms, 47 annular atoms, 48 annular atoms, 49 annular atoms, or 50 annular atoms and the ring contains at least one heteroatom. By way of example and without limitation, these include the heterocycles described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960), each of which is incorporated herein by reference.

Examples of heterocycles include by way of example and not limitation pyridyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, isoindolyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahyclroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, .beta.-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, benzothiophenyl, benzo[c]thiophenyl, purinyl, indazolyl, oxazolyl, benzoxazolyl, isoxazolyl, benzisoxazolyl, benzothiazolyl, pyridinyl, quinolinyl, 1,2,3,4-tetrahydroquinolinyl, 1,2,3,4-tetrahydroisoquinolinyl, isoquinolinyl, pyrazinyl, quinoxalinyl, acridinyl, pyrimidinyl, quinazolinyl, pyridazinyl, dioxanyl, tetrahydropyranyl, aziridinyl, azetidinyl, and morpholinyl.

By way of example and not limitation, carbon-bonded heterocycles may be bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline, or position 1, 3, 4, 5, 6, 7, or 8 of an isoquinoline. In one embodiment, carbon bonded heterocycles may include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles may be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. In one embodiment, nitrogen bonded heterocycles include 1-aziridyl, 1-azetedyl, 1-pyrrolyl, 1-imidazolyl, 1-pyrazolyl, and 1-piperidinyl.

In one embodiment, novel sulfurization reagents, which are bound to solid, polymeric supports, are provided for use in the synthesis of any desired oligonucleotide. Because the sulfurization reagents described herein are bound to solid, polymeric supports, they can generally be removed by simple filtration and washing. Accordingly, purification of the sulfurization reagents is more cost effective and efficient compared to their solution-based counterparts. Moreover, their use also achieves an economical and efficient synthesis and purification of oligonucleotides in solution without resort to expensive purification methods such as chromatography.

As used herein, the term “backbone” denotes the series of covalently or ionically bonded atoms that together create the continuous chain of the polymeric solid support.

As used herein, the term “solid support” refers to any particle, bead, or other surface upon which a sulfur transfer group may be attached.

As used herein, the term “sulfur transfer group” refers to the portion of the sulfurization reagent that is transferred to the oligonucleotide to form a phosphorothioate triester linkage, for example, the —SR₂ group.

In one embodiment, the solid-supported sulfurization reagents described herein comprise one or more moieties according to Formula I:

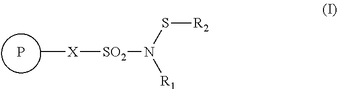

wherein (P) is the backbone of the polymeric solid support and X is a linker between the backbone of the solid support and a sulfonyl group. In one embodiment, the backbone may comprise carbon atoms, oxygen atoms, silicon atoms, or any combination thereof. In another embodiment, the backbone may be formed from the polymerization of any suitable alkene or alkyne compound.

Although the backbone of the polymeric support is depicted in Formula I as having only one sulfur transfer group attached to the back bone of the solid support, it is understood that the polymeric solid support may comprise more than one sulfur transfer group attached to the backbone of the polymeric solid support through multiple linkers.

In one embodiment, the solid support may be any desired organic support. In another embodiment, the solid support may be any sulfonated polymeric support. By way example and without limitation, the organic support may be highly cross-linked polystyrene, grafted copolymers consisting of a low cross-linked polystyrene matrix on which polyethylene glycol (PEG or POE) is grafted (e.g., Tentagel), polyvinylacetate (PVA), a copolymer of polystyrene/divinyl benzene (e.g., Poros), aminopolyethyleneglycol and/or cellulose. In one embodiment, the solid support may be any polystyrene support. In yet another embodiment, the solid support may be a highly cross-linked polystyrene. In one embodiment, the solid support may be a homopolymer, a copolymer, a block-copolymer, or any combination thereof comprising ethylene, propylene, butylenes, styrene, or vinyl monomers. Suitable solid supports include, but are not limited to, solid supports based on poly(4-styrenesulfonic acid), poly(4-styrenesulfonic acid-co-maleic acid), polyanetholesulfonic acid, sulfonated poly(styrene-ran-ethylene), sulfontated polystyrene-block-poly(ethylene-ran-butylene)-block-polystyrene, sulfonated poly(styrene-ran-ethylene), sulfonated polystyrene-block-ethylene-block-polystyrene, sulfonated polystyrene-block-butylene-block-polystytrene, sulfonated polystyrene-block-propylene-block-polystyrene, all of which are commercially available from, for example, Sigma-Aldrich, and their functionalized derivatives. In one embodiment, the solid support may comprise a sulfonated divinylbenzene-based polymer.

In one embodiment, the linker may be a bond. In another embodiment, the linker may be any aliphatic or aromatic linker. Suitable aliphatic linkers may include any alkyl group comprising from 1 carbon atom to 20 carbon atoms and may be linear, branched, or cyclic. By way of example, and without limitation, suitable aliphatic linkers may include methyl, ethyl, propyl, n-butyl, iso-butly, sec-butyl, tert-butyl, pentyl, neo-pentyl, cyclopentyl, hexyl, cyclohexyl, heptyl, cycloheptyl, octyl, cyclooctyl, nonyl, decyl, and adamantyl. The aliphatic linker may optionally be independently substituted at one or more positions with halogen, hydroxyl, alkoxy, aryloxy, substituted aryloxy, amino, acyl, or carboxyl groups. By way of example, and without limitation, the aliphatic linker may be independently substituted at one or more positions with fluoride, chloride, bromide, iodide, hydroxide, methoxy, ethoxy, propoxy, butoxy, pentoxy, phenoxy, amino, methylamino, dimethylamino, ethylyamino, diethylamino, propylamino, and dipropyl amino, and the like.

Suitable aromatic linkers may include any aryl group or heteroaryl group. In one embodiment, a heteroaryl group may be bonded to the S-atom of the sulfonyl group through an annular carbon atom. The aryl and heteroaryl groups may be optionally substituted with one or more aromatic or aliphatic groups. In one embodiment, the aromatic linker may be phenyl, in which case the sulfonyl group may be bonded at either the ortho-, meta-, or para-position. Exemplary aliphatic linkers may include any alkyl group.

The aliphatic, aromation, and heterocyclic linkers may optionally be independently substituted at one or more positions with halogen, hydroxyl, alkoxy, aryloxy, substituted aryloxy, amino, acyl, or carboxyl groups. By way of example, and without limitation, the aliphatic linker may be independently substituted at one or more positions with fluoride, chloride, bromide, iodide, hydroxide, methoxy, ethoxy, propoxy, butoxy, pentoxy, phenoxy, amino, methylamino, dimethylamino, ethylyamino, diethylamino, propylamino, dipropyl amino, and the like.

In one embodiment, R₁ may be any hydrocarbon residue. In another embodiment, the hydrocarbon residue may be substituted, unsubstituted, saturated, unsaturated, cyclic, heterocyclic, aromatic, or heteroaromatic. In one embodiment, R₁ may be a saturated hydrocarbon residue having 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, or 20 carbon atoms. When R₁ is a saturated hydrocarbon residue, it may comprise a linear alkyl group, a branched alkyl group, or a cycloalkyl group. By way of example and without limitation, R₁ may comprise lower alkyl or cycloalkyl groups having 1 carbon atom, 2 carbon atoms, 3 carbon atoms, 4 carbon atoms, 5 carbon atoms, 6 carbon atoms, or 7 carbon atoms such as methyl, ethyl, n-propyl, isopropyl, n-butyl, sec-butyl, tert-butyl, pentyl, hexyl, heptyl, cyclopentyl, cyclohexyl, cycloheptyl, and the like.

In another embodiment, R₁ may be any aromatic hydrocarbon residue. In one embodiment, the aromatic hydrocarbon residue may comprise 3 annular carbon atoms, 4 annular carbon atoms, 5 annular carbon atoms, 6 annular carbon atoms, 7 annular carbon atoms, 8 annular carbon atoms, 9 annular carbon atoms, 10 annular carbon atoms, 11 annular carbon atoms, 12 annular carbon atoms, 13 annular carbon atoms, 14 annular carbon atoms, 15 annular carbon atoms, 16 annular carbon atoms, 17 annular carbon atoms, 18 annular carbon atoms, 19 annular carbon atoms, 20 annular carbon atoms, 21 annular carbon atoms, 22 annular carbon atoms, 23 annular carbon atoms, or 24 annular carbon atoms. Exemplary aromatic hydrocarbon residues include, without limitation, phenyl and naphthyl, which can optionally be substituted with aryl, heteroaryl, alkyl, cycloalkyl, or heterocyclic groups or heterosubstituents such as halogens, amines, ethers, carboxylates, nitro, thiols, sulfonic, and sulfone groups.

In another embodiment, R₁ may be a heterocycle containing one or more annular nitrogen, oxygen or sulfur atoms, which may be bonded to the amino nitrogen through an annular carbon atom. Suitable examples include, without limitation, furanyl, benzofuranyl, isobenzofuranyl, pyrrolyl, indolyl, isoindolyl, thiophenyl, benzothiophenyl, benzo[c]thiophenyl, imidazolyl, benzimidazolyl, purinyl, pyrazolyl, indazolyl, oxazolyl, benzoxazolyl, isoxazolyl, benzisoxazolyl, thiazolyl, benzothioazolyl, pyridinyl, quinolinyl, isoquinolinyl, pyrazinyl, quinoxalinyl, acridinyl, pyrimidinyl, quinazolinyl, pyridazinyl, or cinnolinyl. These heterocycles may optionally be substituted with aryl, heteroaryl, alkyl, cycloalkyl, or heterocyclic groups or heterosubstituents such as halogens, amines, ethers, carboxylates, nitro, thiols, sulfonic, and sulfone groups.

In one embodiment, R₂ may comprise an alkyl or aryl group. In another embodiment, R₂ may comprise a substituted or unsubstituted phenyl group. In still another embodiment, R₂ may comprise a phenyl group substituted with a halogen or an alkyl group containing 1 carbon atom, 2 carbon atoms, 3 carbon atoms, 4 carbon atoms, 5 carbon atoms, 6 carbon atoms, 7 carbon atoms, or 8 carbon atoms. Exemplary phenyl groups include, without limitation, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 2-iodophenyl, 3-iodophenyl, 4-iodophenyl, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-isopropylphenyl, 3-isopropylphenyl, 4-isopropylphenyl, 2-n-butylphenyl, 3-n-butylphenyl, 4-n-butylphenyl, 2-sec-butylphenyl, 3-sec-butylphenyl, 4-sec-butylphenyl, 2-iso-butylphenyl, 3-isobutylphenyl, 4-isobutylphenyl, 2-tert-butylphenyl, 3-tert-butylphenyl, 4-tert-butylphenyl, 2,4-dimethylphenyl, 2,3-dimethylphenyl, and 3,4-dimethylphenyl.

In one embodiment, R₂ may be a methyleneacyloxy group. As used herein, a “methyleneacyloxy group” has the formula —CH₂—O—C(O)—R₇, wherein R₇ is a hydrocarbon residue having 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, or 20 carbon atoms. In one embodiment, the hydrocarbon residue R₇ may be saturated, unsaturated, cyclic, heterocyclic, aromatic, or heteroaromatic. In the case of a saturated hydrocarbon residue, R₇ may be linear, branched, or cyclic. In yet another embodiment, R₇ may be a lower alkyl or lower cycloalkyl residue. By way of example and without limitation, R₇ may by methyl, ethyl, n-propyl, isopropyl, n-butyl, sec-butyl, tert-butyl, pentyl, cyclopentyl, hexyl, and cyclohexyl. In the case of an aromatic or heteroaromatic hydrocarbon residue, R₇ may be an aromatic or heteroaromatic system having 3 annular atoms, 4 annular atoms, 5 annular atoms, 6 annular atoms, 7 annular atoms, 8 annular atoms, 9 annular atoms, 10 annular atoms, 11 annular atoms, 12 annular atoms, 13 annular atoms, or 14 annular atoms. By way of example and without limitation, suitable aromatic residues may be phenyl, naphthyl, and anthracyl groups. These aromatic residues may be optionally substituted with aryl, heteroaryl, alkyl, cycloalkyl, heterocyclic, halogen, amino, ether, ester, carboxylate, nitro, thiol, sulfonic, and sulfone groups. In the case of a heterocyclic residue, R₇ may be a heterocycle containing at least one annular nitrogen, oxygen or sulfur atom. By way of example and without limitation, the heterocyclic residue may be furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, thiazole, benzothiazole, pyridine, quinoline, isoquinoline, pyrazine, quinoxaline, acridine, pyrimidine, quinazoline, pyridazine, and cinnoline.

In a further embodiment, R₂ may be a methylene carbonate group. As used herein, a “methylene carbonate group” has the formula —CH₂—O—C(O)—OR₈, wherein R₈ may be any alkyl, cycloalkyl, aryl, or heteroaryl. Suitable alkyls and cycloalkyls include lower alkyls and lower cycloalkyls. By way of example and without limitation, R₈ may be methyl, ethyl, n-propyl, isopropyl, n-butyl, sec-butyl, tert-butyl, pentyl, cyclopentyl, hexyl, and cyclohexyl. In another embodiment, R₈ may be an aromatic system having 3 annular carbon atoms, 4 annular carbon atoms, 5 annular carbon atoms, 6 annular carbon atoms, 7 annular carbon atoms, 8 annular carbon atoms, 9 annular carbon atoms, 10 annular carbon atoms, 11 annular carbon atoms, 12 annular carbon atoms, 13 annular carbon atoms, or 14 annular carbon atoms such as phenyl, naphthyl, and anthracyl. In an additional embodiment, R₈ may be a heteroaromatic system having 1 annular carbon atom, 2 annular carbon atoms, 3 annular carbon atoms, 4 annular carbon atoms, 5 annular carbon atoms, 6 annular carbon atoms, 7 annular carbon atoms, 8 annular carbon atoms, 9 annular carbon atoms, 10 annular carbon atoms, 11 annular carbon atoms, 12 annular carbon atoms, 13 annular carbon atoms, or 14 annular carbon atoms and at least one annular nitrogen, oxygen or sulfur atom such as furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, thiazole, benzothiazole, tetrazole, pyridine, quinoline, isoquinoline, pyrazine, quinoxaline, acridine, pyrimidine, quinazoline, pyridazine, and cinnoline. In yet another embodiment, R₈ may be independently substituted with one or more aryl, heteroaryl, alkyl, cycloalkyl, heterocyclic, halogen, amino, ether, ester, carboxylate, nitro, thiol, sulfonic, and sulfone groups.

In another embodiment, R₂ may be saturated, unsaturated, cyclic, heterocyclic, aromatic, or heteroaromatic. In the case of a saturated hydrocarbon residue, R₂ may be linear, branched, or cyclic. In yet another embodiment, R₂ may be a lower alkyl or lower cycloalkyl residue. As used herein, “lower alkyl” and “lower cycloalkyl” denotes an alykyl and cycloalkyl, respectively, having from one to seven carbon atoms in the residue. By way of example and without limitation, the saturated hydrocarbon residue may by methyl, ethyl, n-propyl, isopropyl, n-butyl, sec-butyl, tert-butyl, pentyl, cyclopentyl, hexyl, and cyclohexyl. In the case of an aromatic or heteroaromatic hydrocarbon residue, R₂ may be an aromatic or heteroaromatic system having from 1 annular carbon atom, 2 annular carbon atoms, 3 annular carbon atom, 4 annular carbon atom, 5 annular carbon atoms, 6 annular carbon atoms, 7 annular carbon atoms, 8 annular carbon atoms, 9 annular carbon atoms, 10 annular carbon atoms, 11 annular carbon atoms, 12 annular carbon atoms, or 14 annular carbon atoms. By way of example and without limitation, suitable aromatic residues may be phenyl, naphthyl, and anthracyl groups. These aromatic residues may be optionally substituted with one or more aryl, heteroaryl, alkyl, cycloalkyl, heterocyclic, halogen, amino, ether, ester, carboxylate, nitro, thiol, sulfonic, and sulfone groups. In one embodiment, R₂ may be 2-chlorophenyl, 3-chlorophenyl, 4-chlorophenyl, 2-bromophenyl, 3-bromophenyl, 4-bromophenyl, 2-iodophenyl, 3-iodophenyl, 4-iodophenyl, 2-fluorophenyl, 3-fluorophenyl, 4-fluorophenyl, 2-nitrophenyl, 3-nitrophenyl, or 4-nitrophenyl.

In still another embodiment, R₂ may be a methylene carbamate group. As used herein, a “methylene carbamate group” has the formula —CH₂—O—C(O)—NR₉R₁₀, wherein R₉ and R₁₀ may independently be hydrogen, alkyl, cycloalkyl, aryl, or heteroaryl. Suitable alkyls and cycloalkyls include lower alkyls and lower cycloalkyls. By way of example and without limitation, R₉ and R₁₀ may be methyl, ethyl, n-propyl, isopropyl, n-butyl, sec-butyl, tert-butyl, pentyl, cyclopentyl, hexyl, and cyclohexyl. Suitable aryls may be any aromatic system having from 6 to 14 carbon atom such as phenyl, naphthyl, and anthracyl. Suitable heteroeryls may be any heteroaromatic system having 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, or 14 carbon atoms and at least one annular nitrogen, oxygen or sulfur atom such as furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, thiazole, benzothiazole, pyridine, quinoline, isoquinoline, pyrazine, quinoxaline, acridine, pyrimidine, quinazoline, pyridazine, and cinnoline. In yet another embodiment, R₉ and R₁₀ may be independently substituted with one or more aryl, heteroaryl, alkyl, cycloalkyl, heterocyclic, halogen, amino, ether, ester, carboxylate, nitro, thiol, sulfonic, and sulfone groups. In one embodiment, R₉ and R₁₀ may be methyl or ethyl to form a N,N-dimethyl amino group or a N,N-diethyl amino group. In another embodiment, R₉ and R₁₀ form a N-heterocyclic ring having 3 annular atoms, 4 annular atoms, 5 annular atoms, 6 annular atoms, 7 annular atoms, or 8 annular atoms, wherein the ring may optionally comprise additional nitrogen, oxygen and sulfur atoms. In yet another embodiment, R₉ and R₁₀ form an N-piperidyl group or N-pyrrolidyl group.

In one embodiment, the solid-supported sulfurization reagent has the general structure according to Formula II, wherein the linker X in Formula I is a phenyl group:

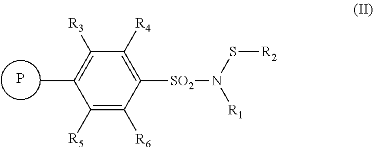

While Formula II is depicted as having a para-substituted benzene linker, it is understood that ortho- and meta-substituted benzene linkers according to Formulas III and IV are also within the scope of this disclosure. It is also understood that more than one linker may be attached to the backbone of the polymeric solid support.

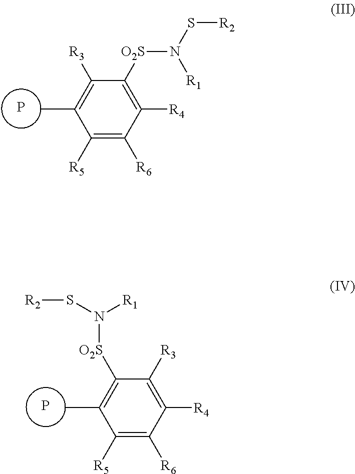

In one embodiment, R₃, R₄, R₅ and R₆ may independently be hydrogen, fluoride, chloride, bromide, iodide, hydroxyl, alkyl, cycloalkyl, aryl, alkoxy, aryloxy, heteroaryl, heterocyclic, amino, ether, ester, acyl, carboxylate, nitro, thiol, sulfonic acid, sulfonic acid derivative, and sulfone. In one embodiment, R₃, R₄, R₅ and R₆ are hydrogen. In a further embodiment, one or more of R₃, R₄, R₅ and R₆ are methoxy, epoxy, amino, and dimethylamino.

In one embodiment, the solid-supported sulfurization reagent may comprise one or more of the following moieties:

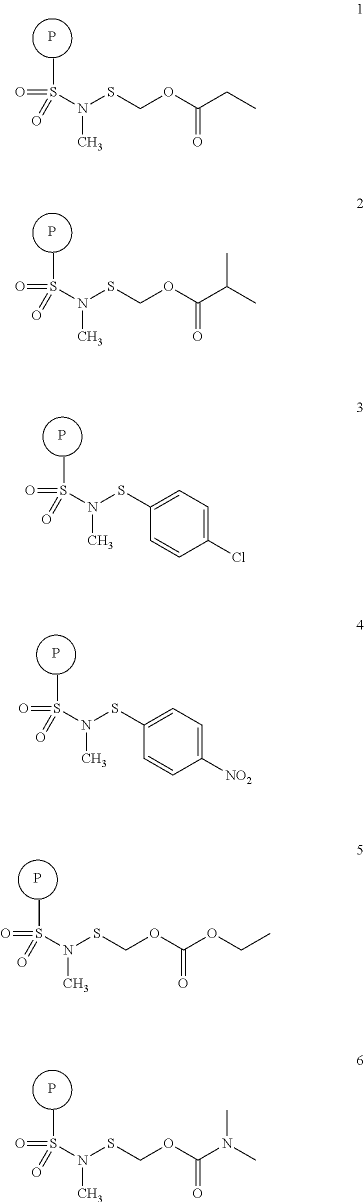

In another embodiment, the solid-supported sulfurization reagent may comprise one or more of the following moieties:

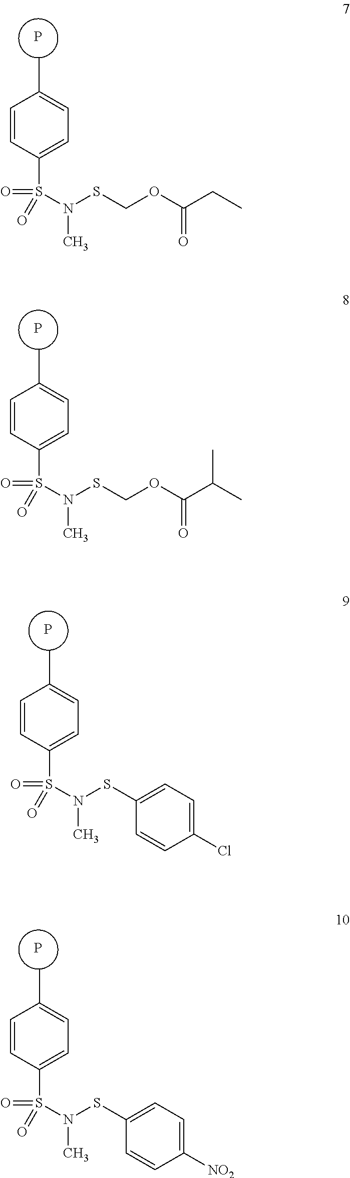

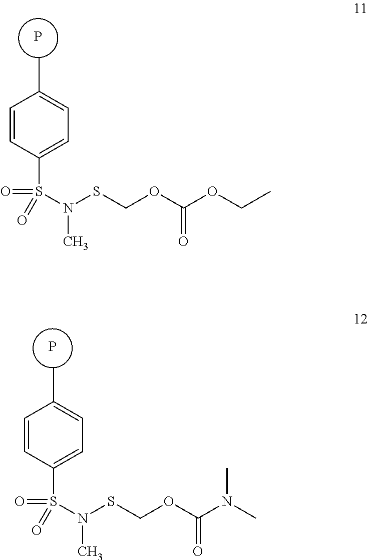

In still another embodiment, the solid-supported sulfurization reagent may comprise one or more of the following polystyrene-based moieties:

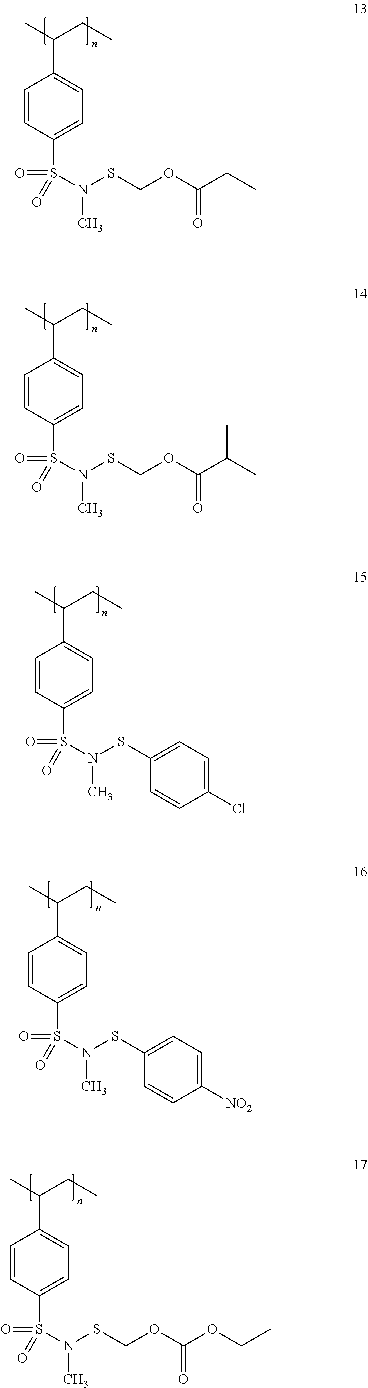

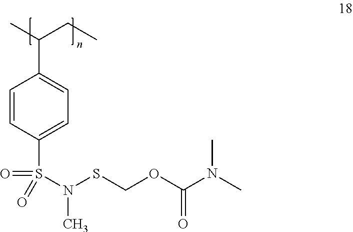

wherein n is an integer of 1 or greater.

In one embodiment, the number of sulfonated styrene units may be from about 5% to about 90%. In another embodiment, the number of sulfonated styrene units may be from about 15% to about 60%. In yet another embodiment, the number of sulfonated styrene units may be from about 25% to about 40%. In a further embodiment, the number of sulfonated styrene units may be about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%.

In another embodiment, the number of sulfonated styrene units containing a sulfur transfer group may be from about 5% to 100%. In still another embodiment, the number of sulfonated styrene units containing a sulfur transfer group may be about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100%.

As used herein, the term “load” refers to the molar equivalents of the sulfur transfer groups in 1 gram of the solid-supported sulfurization reagent.

In one embodiment, the polymeric solid support may have a loading from about 0.1 mmol to about 5.0 mmol of the sulfur transfer group per 1 gram of the solid-supported sulfurization reagent. In a further embodiment, the polymeric solid support may have a loading of about 0.1 mmol, about 0.2 mmol, about 0.3 mmol, about 0.4 mmol, about 0.5 mmol, about 0.6 mmol, about 0.7 mmol, about 0.8 mmol, about 0.9 mmol, about 1.0 mmol, about 1.1 mmol, about 1.2 mmol, about 1.3 mmol, about 1.4 mmol, about 1.5 mmol, about 1.6 mmol, about 1.7 mmol, about 1.8 mmol, about 1.9 mmol, about 2.0 mmol, about 2.1 mmol, about 2.2 mmol, about 2.3 mmol, about 2.4 mmol, about 2.5 mmol, about 2.6 mmol, about 2.7 mmol, about 2.8 mmol, about 2.9 mmol, about 3.0 mmol, about 3.1 mmol, about 3.2 mmol, about 3.3 mmol, about 3.4 mmol, about 3.5 mmol, about 3.6 mmol, about 3.7 mmol, about 3.8 mmol, about 3.9 mmol, about 4.0 mmol, about 4.1 mmol, about 4.2 mmol, about 4.3 mmol, about 4.4 mmol, about 4.5 mmol, about 4.6 mmol, about 4.7 mmol, about 4.8 mmol, about 4.9 mmol, or about 5.0 mmol of the sulfur transfer group relative to about 1 gram of the solid-supported sulfurization reagent.

Synthesis of Sulfurization Reagents

In one embodiment, the solid-supported sulfurization reagents described herein may be synthesized according to a general process comprising the steps set forth in Scheme I. According to Scheme I, the synthesis of the solid-supported sulfurization reagents comprises (i) reacting a solid-supported sulfonyl chloride 19 with an amine NH₂Ri, wherein R₁ is as previously defined, to yield the corresponding sulfonamide compound 20, and (ii) reacting the sulfonamide compound 20 with any desired sulfenyl chloride R₂SC1, wherein R₂ is as previously defined, to yield the corresponding solid-supported sulfurization reagent 21.

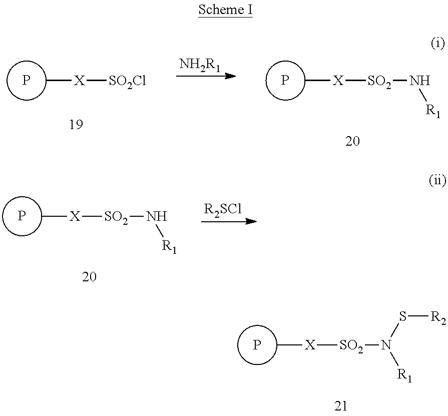

In one embodiment, the solid-supported sulfonyl chloride may be any desired sulfonyl chloride attached to any suitable solid support. In another embodiment, the solid-supported sulfonyl chloride may be derived by functionalization of the sulfonated solid supports previously described. By way of example and without limitation, suitable solid-supported sulfonyl chlorides include the 70-90 mesh sulfonyl chlorides having about a 2.5 mmol/g load to about a 3.0 mmol/g load and the 100-200 mesh with about a 1.0 to about a 2.0 mmol/g load polymer-bound sulfonyl chlorides, which are about 1% to about 8.5% cross-linked with divinylbenzene and commercially available from, for example, Sigma-Aldrich. Additionally, solid-supported benzenesulfonyl chlorides, which are also commercially available from, for example, Sigma-Aldrich, are suitable.

Because the sulfurization reagents described herein are bound to solid supports, they can be efficiently removed from the reaction mixture containing other sulfurization by-products. Consequently, the sulfurization reagents described herein can be obtained with acceptable purity by filtration and washing, unlike solution phase sulfurization reagents, which generally requires time-consuming and expensive purification techniques such as chromatography just prior to use in oligonucleotide synthesis.

In one embodiment, solid-supported sulfonyl chloride 19 may comprise a polystyrene-based sulfonyl chloride moiety having the following structure:

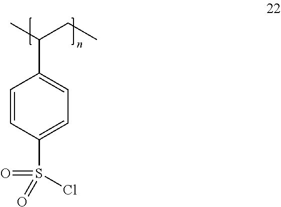

wherein n is an integer of 1 or higher and represents the number of polystyrene-based sulfonyl chloride moieties attached to the polymeric backbone. While this embodiment depicts a solid-supported sulfonyl chloride reagent comprising a para-substituted benzene linker, solid-supported sulfonyl chloride reagents comprising meta- and ortho-substituted benzene linkers are also understood to be within the scope of the present disclosure.

In another embodiment, solid-supported sulfonamide 20 may comprise a polystyrene-based sulfonamide moiety having the following structure:

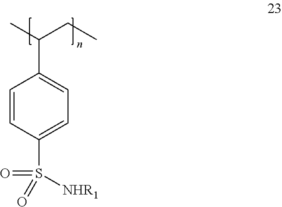

wherein n is an integer of 1 or higher and represents the number of polystyrene-based sulfonyl chloride moieties attached to the polymeric backbone and R1 is as previously defined. While 22 is depicted as a solid-supported sulfonamide, wherein the sulfonamide group is attached to the para-position of the phenyl ring, one of ordinary skill in the art would understand that the sulfonamide group may be also be attached to either the ortho or meta-positions.

In one embodiment, step (i) of the synthesis of the sulfurization reagent may be carried out in any suitable organic solvent. In another embodiment, step (i) of the synthesis of the sulfurization reagent may be carried out in an aprotic, organic solvent. Exemplary polar, aprotic solvents include, without limitation, tetrahydrofuran, acetone, acetonitrile, dimethylsulfoxide, dimethylacetamide, ethyl acetate, methyl acetate, hexamethylphosphoramide, N-methylpyrrolidone, 1,3-dimethyl-2-imidazolidinone, dichloromethane, dichloroethane and the like. In another embodiment, step (i) of the synthesis of the sulfurization reagent may be carried out in an aromatic solvent such as toluene, xylene, benzene, and the like. In a further embodiment, step (i) of the synthesis of the sulfurization reagent may be carried out in any desired solvent that is a liquid at about room temperature or below.

In one embodiment, step (i) of the synthesis of the sulfurization reagent may be carried out at any suitable reaction temperature. In another embodiment, step (i) of the synthesis of the sulfurization reagent may be carried out at a temperature from about −80° C. to about 60° C. In still another embodiment, step (i) of the synthesis of the sulfurization reagent may be carried out at a temperature of about −80° C., about −75° C., about −70° C., about −65° C., about −60° C., about −55° C., about −50° C., about −45° C., about −40 C, about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., or about 60° C.

In one embodiment, step (ii) of the synthesis of the sulfurization reagent may be carried out in any suitable organic solvent. In another embodiment, step (ii) of the synthesis of the sulfurization reagent may be carried out in an aprotic, organic solvent. Exemplary polar, aprotic solvents include, without limitation, tetrahydrofuran, acetone, acetonitrile, dimethylsulfoxide, dimethylacetamide, ethyl acetate, methyl acetate, hexamethylphosphoramide, N-methylpyrrolidone, 1,3-dimethyl-2-imidazolidinone, dichloromethane, dichloroethane, and the like. In another embodiment, step (ii) of the synthesis of the sulfurization reagent may be carried out in an aromatic solvent such as toluene, xylene, benzene, and the like. In a further embodiment, step (ii) of the synthesis of the sulfurization reagent may be carried out in any desired solvent that is a liquid at about room temperature or below.

In one embodiment, step (ii) of the synthesis of the sulfurization reagent may be carried out at any suitable reaction temperature. In another embodiment, step (ii) of the synthesis of the sulfurization reagent may be carried out at a temperature from about −80° C. to about 60° C. In still another embodiment, step (ii) of the synthesis of the sulfurization reagent may be carried out at a temperature of about −80° C., about −75° C., about −70° C., about −65° C., about −60° C., about −55° C., about −50° C., about −45° C., about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., or about 60° C.

Synthesis of Oligonucleotides

As used herein, the term “oligonucleotides” denotes an oligomer of nucleoside monomeric units comprising sugar units connected to nucleobases, wherein the nucleoside monomeric units are connected by internucleotide bonds. An “internucleotide bond” refers to a chemical linkage between two nucleoside moieties, such as the phosphodiester linkage typically present in natural nucleic acids or other linkages typically present in synthetic nucleic acids and nucleic acid analogues. For example, and without limitation, an internucleotide bond may include a phospho or phosphite group and linkages where one or more oxygen atoms of the phospho or phosphite group are either modified with a substituent or replaced with another atom, such as a sulfur atom or the nitrogen atom of a mono- or di-alkyl amino group. Typical internucleotide bonds are diesters of phosphoric acid or its derivatives, such as phosphates, thiophosphates, dithiophosphates, phosphoramidates, and thiophosphoramidates.

The term “nucleoside” denotes a compound consisting of a nucleobase connected to a sugar. Sugars may be based on a furanose ring, such as ribose, 2′-deoxyribose, or a non-furanose ring, such as cyclohexenyl, anhydrohexitol, and morpholino. The modifications, substitutions and positions of the nucleoside sugar indicated hereinafter are discussed with reference to a furanose ring, but the same modifications and positions also apply to analogous positions of other sugar rings. The sugar may be additionally modified. By way of example and without limitation, the sugar may be modified at its 2′- or 3′-position. In one embodiment, the 2′-position of a furanosyl sugar ring may be substituted with hydrogen; hydroxy; an alkoxy group such as methoxy, ethoxy, allyloxy, isopropoxy, butoxy, isobutoxy, methoxyethyl; an aryloxy group such as phenoxy; azido; amino; alkylamino; fluoro; chloro and bromo. In another embodiment, the furanosyl sugar ring may be modified to include cyclic groups formed by the 2′-4′-positions or the 3′-4′-positions in the furanosyl sugar ring. Further modifications of the furanosyl sugar ring may also include substitution of the ring 4′-O with —S—, —CH₂—, —NR—, —CHF— or —CF₂—.

The term “nucleobase” denotes in particular a nitrogen-containing heterocyclic moiety capable of pairing with a complementary nucleobase or nucleobase analogue. Naturally occurring nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, and fluorinated bases. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2’:4,5]pyrrolo[2,3-d]pyrimidin-2-one). Other potentially suitable bases include universal bases, hydrophobic bases, promiscuous bases and size-expanded bases.

Generally, the term “oligonucleotide” refers to a nucleoside subunit polymer having from about 2 contiguous subunits to about 100 contiguous subunits. In one embodiment, the oligonucleotide may comprise from about 2 contiguous subunits to about 50 contiguous subunits. In another embodiment, the oligonucleotide may comprise from about 4 contiguous subunits to about 40 contiguous subunits. In yet another embodiment, the oligonucleotide may comprise from about 6 contiguous subunits to about 30 contiguous subunits. In a further embodiment, the oligonucleotide may comprise 2 contiguous subunits, 3 contiguous subunits, 4 contiguous subunits, 5 contiguous subunits, 6 contiguous subunits, 7 contiguous subunits, 8 contiguous subunits, 9 contiguous subunits, 10 contiguous subunits, 11 contiguous subunits, 12 contiguous subunits, 13 contiguous subunits, 14 contiguous subunits, 15 contiguous subunits, 16 contiguous subunits, 17 contiguous subunits, 18 contiguous subunits, 19 contiguous subunits, 20 contiguous subunits, 21 contiguous subunits, 22 contiguous subunits, 23 contiguous subunits, 24 contiguous subunits, 25 contiguous subunits, 26 contiguous subunits, 27 contiguous subunits, 28 contiguous subunits, 29 contiguous subunits, 30 contiguous subunits, 31 contiguous subunits, 32 contiguous subunits, 33 contiguous subunits, 34 contiguous subunits, 35 contiguous subunits, 36 contiguous subunits, 37 contiguous subunits, 38 contiguous subunits, 39 contiguous subunits, 40 contiguous subunits, 41 contiguous subunits, 42 contiguous subunits, 43 contiguous subunits, 44 contiguous subunits, 45 contiguous subunits, 46 contiguous subunits, 47 contiguous subunits, 48 contiguous subunits, 49 contiguous subunits, 50 contiguous subunits, 51 contiguous subunits, 52 contiguous subunits, 53 contiguous subunits, 54 contiguous subunits, 55 contiguous subunits, 56 contiguous subunits, 57 contiguous subunits, 58 contiguous subunits, 59 contiguous subunits, 60 contiguous subunits, 61 contiguous subunits, 62 contiguous subunits, 63 contiguous subunits, 64 contiguous subunits, 65 contiguous subunits, 66 contiguous subunits, 67 contiguous subunits, 68 contiguous subunits, 69 contiguous subunits, 70 contiguous subunits, 71 contiguous subunits, 72 contiguous subunits, 73 contiguous subunits, 74 contiguous subunits, 75 contiguous subunits, 76 contiguous subunits, 77 contiguous subunits, 78 contiguous subunits, 79 contiguous subunits, 80 contiguous subunits, 81 contiguous subunits, 82 contiguous subunits, 83 contiguous subunits, 84 contiguous subunits, 85 contiguous subunits, 86 contiguous subunits, 87 contiguous subunits, 88 contiguous subunits, 89 contiguous subunits, 90 contiguous subunits, 91 contiguous subunits, 92 contiguous subunits, 93 contiguous subunits, 94 contiguous subunits, 95 contiguous subunits, 96 contiguous subunits, 97 contiguous subunits, 98 contiguous subunits, 99 contiguous subunits, or 100 contiguous subunits.

The nucleoside subunits can be joined by a variety of intersubunit linkages. The term “oligonucleotide” also includes any oligonucleotide modified by methods known to one skilled in the art, such as modifications to the sugar backbone (e.g., oxygen and sulphur substitutions such as phosphoramidate, phosphorodithioate), the sugar (e.g., 2′-substitutions such as 2′-F, 2′-OMe), the base, and the 3′- and 5′-termini. In various embodiments, the oligonucleotide may contain nucleosides such as ribonucleosides, 2′-deoxyribonucleosides, 2′-substituted ribonucleosides, 2′-4′-locked-ribonucleosides, 3′-amino-ribonucleosides, and 3′-amino-2′-deoxyribonucleosides.

In one embodiment, the solid-supported sulfurization reagents described herein may be used in the synthesis of any desired oligonucleotide using the H-phosphonate method. Generally, this method of oligonucleotide synthesis comprises at least: (a) a coupling step, wherein a phosphorus internucleotide linkage is formed between two reactants selected from nucleotides and oligonucleotides; and (b) a sulfurization step, wherein a sulfurization reagent described herein is employed to sulfurize the phosphorus internucleotide linkage, as generally set forth in Scheme II.

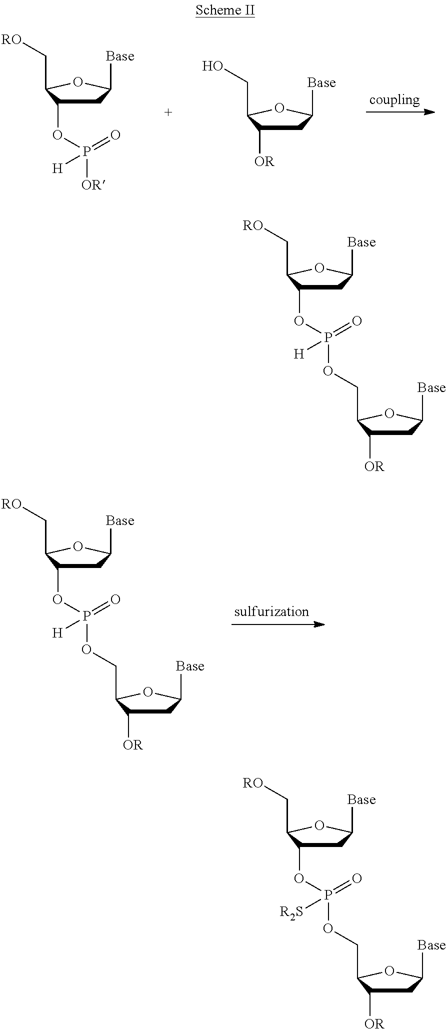

wherein OR′ is a leaving group and OR is a protecting group.

The coupling step may comprise forming a H-phosphonate diester bond by coupling an H-phosphonate monoester salt with a protected nucleoside or oligonucleotide having a free hydroxyl group. In one embodiment, the coupling may be carried out in solution using an aprotic organic solvent. Suitable solvents include halogenated solvents such as chlorinated hydrocarbons and nitrogen-containing solvents such as N-heterocyclic solvents, acetonitrile, pyridine, and the like. The reaction to form an H-phosphonate diester may be accomplished by use of a carboxylic acid halide such as pivaloyl chloride and the like.

In one embodiment, the coupling step may be carried out at a temperature from about −50° C. to about 60° C. In another embodiment, the coupling step is carried out at a temperature from about −20° C. to about 40° C. In a further embodiment, the coupling step is carried out at a temperature from about 0° C. to about 20° C. In still another embodiment, the coupling step is carried out at a temperature of about −50° C., about −45° C., about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., or about 60° C.

In another embodiment, the liquid reaction medium used in the coupling step may contain from about 2% to about 75% by weight of the H-phosphonate oligonucleotide relative to the total weight of the reaction medium. In a further embodiment, the liquid reaction medium used in the coupling step may contain from about 10% to about 65% by weight of the H-phosphonate oligonucleotide relative to the total weight of the reaction medium. In yet another embodiment, the liquid reaction medium used in the coupling step may contain from about 20% to about 55% by weight of the H-phosphonate oligonucleotide relative to the total weight of the reaction medium. In still another embodiment, the liquid reaction medium used in the coupling step may contain about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, or about 65% by weight of the H-phosphonate oligonucleotide relative to the total weight of the reaction medium.

In one embodiment, the sulfurization step may be carried out in any desired solvent so long as the oligonucleotide is sufficiently soluble. In another embodiment, the sulfurization step may be carried out in a polar, aprotic organic solvent such as a halogenated hydrocarbon solvent and nitrogen-containing solvents and any combinations thereof. Exemplary solvents include, but are not limited to, halogenated hydrocarbons such as dichloromethane and N-heterocyclic solvents such as pyridine. In one embodiment, the sulfurization step may be carried out in a mixture comprising pyridine and dichloromethane. In another embodiment, the sulfurization reaction may be carried out dimethyl acetamide (DMA), N,N-dimethylformamide (DMF), N-methylpyrrolidone (NMP), dimethylsulfoxide (DMSO), pyridine, acetonitrile, chlorinated hydrocarbons, dichloromethane, chloroform, ethyl acetate, isopropyl acetate, and the like.

In one embodiment, the sulfurization step is carried out at a temperature from about −50° C. to about 60° C. In another embodiment, the sulfurization step is carried out at a temperature from about 0° C. to about 20° C. In yet another embodiment, the sulfurization step is carried out at a temperature from about −50° C., about −45° C., about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., or about 60° C.

In one embodiment, the sulfurization step employs a solid-supported sulfurization reagent having a molar ratio of sulfur transfer groups relative to the amount of internucleotide linkages of from about 1 to about 4. In another embodiment, the sulfurization step employs a sulfurization reagent having a molar ratio of sulfur transfer groups relative to the amount of internucleotide linkages of from about 1.5 to about 4. In yet another embodiment, the sulfurization step employs a sulfurization reagent having a molar ratio of sulfur transfer groups relative to the amount of internucleotide linkages of from about 2 to about 4. In still another embodiment, the sulfurization step employs a sulfurization reagent having a molar ratio of sulfur transfer groups relative to the amount of internucleotide linkages of about 1, about 1.1, about 1.2, about, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0.

In one embodiment, the sulfurization step may activate the H-phosphonate diester with an activator such as a base. Exemplary activators include, but are not limited to, alkylamines, tertiary alkylamines, diisopropylethylamine, and the like.

In one embodiment, the coupling and sulfurization steps may be repeated as desired after each 3′- or 5′-deprotection of the sulfurized oligonucleotide. When more than one sulfurization step is performed, each sulfurization step may be performed independently using any solid-supported sulfurization reagent described herein such that the desired oligonucleotide may contain one type or more than one type of a sulfur transfer group attached to the phosphorus atoms of the oligonucleotide backbone.

In one embodiment, the H-phoshonate diester formed in the coupling step may be isolated and subsequently sulfurized in the sulfurization step. In another embodiment, the H-phosphonate diester generated in the coupling step may be sulfurized in the sulfurization step without isolation. In yet another embodiment, sulfurization of the H-phosphonate diester formed in the coupling step may be carried out in situ by addition of a solution of the solid-supported sulfurization reagent directly to the reaction medium of the coupling step. In still another embodiment, sulfurization of the H-phosphonate diester formed in the coupling step may be carried out after purification of the H-phosphonate diester from the reaction mixture.

Regeneration of the Solid-Supported Sulfurization Reagent

By using the sulfurization reagents described herein, sulfurization of the phosphonate hydrogen atom during oligonucleotide synthesis yields a sulfurized oligonucleotide and produces solid-supported sulfonamide 24 as a by-product. Accordingly, it may be desirable to regenerate the sulfurization reagent 25 using the solid-supported sulfonamide by-product, as generally shown in Scheme III.

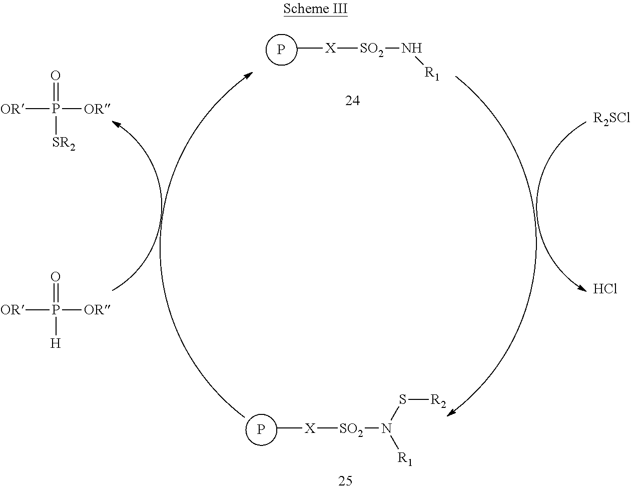

wherein OR′—P—OR″ comprises the sugar backbone of an oligonucleotide.

In a typical cycle, the solid-supported sulfurization reagent 25 reacts with the H-phosphonate diester, producing the solid-supported sulfonamide 24 by-product. The sulfur transfer from the solid-supported sulfurization reagent 25 to the oligonucleotide occurs via metathesis of the N—SR₂ of the solid-supported sulfurization reagent and the P—H bond of the H-phosphonate diester. Because this by-product of the sulfurization reaction is solid, it can easily be removed from the solution containing the sulfurized oligonucleotide by simple filtration and washing, allowing for an efficient method for purifying the sulfurized oligonucleotide. The solid by-product can then be collected, washed, and used to regenerate the solid-supported sulfurization reagent 25.

In one embodiment, the solid-supported sulfurization reagent 25 may be regenerated from the solid-supported sulfonamide by-product 24 any number of times so long as the regenerated solid-supported sulfurization reagent has desired reactivity and does not introduce an undesired amount of impurities.

Purification of the Oligonucleotides

The solid-supported sulfurization reagents disclosed herein are useful for a method of purifying an oligonucleotide having at least one P—S—R linkage as described herein. In one embodiment, the method comprises precipitating the oligonucleotide. In another embodiment, the method further comprises extraction of the oligonucleotide (e.g., from solid material recovered from the precipitation step) with a solvent. Suitable solvents for extraction include, but are not limited to, any desired polar organic solvent or combinations thereof.

In one embodiment, the purification may be effectively accomplished by a combination of precipitation and extraction techniques of the protected oligonucleotide obtained according to the described method. The exact conditions of precipitation depend on the given sequence and length of the oligonucleotide. In one embodiment, the precipitation method generally comprises (a) dissolving the oligonucleotide in a polar organic solvent and (b) adding a non-polar organic solvent until the solution becomes turbid.

In one embodiment, the oligonucleotides made using the solid-supported sulfurization reagents described herein may generally be isolated and purified by precipitation.

In one embodiment, the solvent used to dissolve the oligonucleotide in step (a) may be halogenated hydrocarbons such as methylene chloride and chloroform, nitrogen containing solvents such as acetonitrile and pyridine, and carbonyl-containing solvents such as acetone, and the like.

In one embodiment, in step (a), a solvent volume may be used ranging from about 0.5(n+1) mL to about 2.0(n+1) mL, wherein n is the number of millimoles of the phosphorothioate triester linkages. In another embodiment, in step (a) a solvent volume may be used of about 1.0(n+1) mL. In still another embodiment, in step (a), a solvent volume may be about 0.5(n+1) mL, about 0.55(n+1) mL, about 0.6(n+1) mL, about 0.65(n+1) mL, about 0.7(n+1) mL, about 0.75(n+1) mL, about 0.8(n+1) mL, about 0.85(n+1) mL, about 0.9(n+1) mL, about 0.95(n+1) mL, about 1.0(n+1) mL, about 1.05(n+1) mL, about 1.1(n+1) mL, about 1.15(n+1) mL, about 1.2(n+1) mL, about 1.25(n+1) mL, about 1.3(n+1) mL, about 1.35(n+1) mL, about 1.4(n+1) mL, about 1.45(n+1) mL, about 1.5(n+1) mL, about 1.55(n+1) mL, about 1.6(n+1) mL, about 1.65(n+1) mL, about 1.7(n+1) mL, about 1.75(n+1) mL, about 1.8(n+1) mL, about 1.85 (n+1) mL, about 1.9(n+1) mL, about 1.95 (n+1) mL, and about 2.0(n+1) mL.

In one embodiment, the solution of the oligonucleotide is treated with a non-polar organic solvent, including but not limited to hydrocarbons such as alkane solvents like pentane and hexane; ether solvents such as methyl-tert-butyl ether (MTBE); mixtures thereof such as hexane/MTBE mixtures until the solution becomes turbid; and the like. In yet another embodiment, the turbid solution is subsequently treated with a precipitation aid. Suitable precipitation aids include, but are not limited to, inert porous solids such as Celite, charcoal, and wood cellulose and chromatography stationary phases such as silica or alumina.

In one embodiment, the precipitation aid may generally be used in an amount ranging from about 0.25(n+1) grams to about 1.5(n+1) grams, where n is the number of millimoles of phosphorothioate triester linkages. In another embodiment, the precipitation aid may generally be used in an amount of about 0.75(n+1) grams. In yet another embodiment, the precipitation aid may generally be used in an amount of about 0.25(n+1) grams, about 0.30(n+1) grams, about 0.35(n+1) grams, about 0.40(n+1) grams, about 0.45(n+1) grams, about 0.50(n+1) grams, about 0.55(n+1) grams, about 0.60(n+1) grams, about 0.65(n+1) grams, about 0.70(n+1) grams, about 0.75(n+1) grams, about 0.80(n+1) grams, about 0.85(n+1) grams, about 0.90(n+1) grams, about 1.00(n+1) grams, about 1.05(n+1) grams, about 1.10(n+1) grams, about 1.15(n+1) grams, about 1.20(n+1) grams, about 1.25(n+1) grams, about 1.30(n+1) grams, about 1.35(n+1) grams, about 1.40(n+1) grams, about 1.45(n+1) grams, and about 1.50(n+1) grams.

In one embodiment, after addition of the precipitation aid, the mixture may be treated with a second fraction of a non-polar organic solvent as described above. In one embodiment, the volume of the second fraction may range from about 1(n+1) mL to about 4(n+1) mL, wherein n is the number of millimoles of phosphorothioate triester linkages. In another embodiment, the volume of the second fraction may be about 2.0(n+1) mL. In yet another embodiment, the volume of the second fraction may about 1.0(n+1) mL, about 1.1(n+1) mL, about 1.2(n+1) mL, about 1.3(n+1) mL, about 1.4(n+1) mL, about 1.5(n+1) mL, about 1.6(n+1) mL, about 1.7(n+1) mL, about 1.8(n+1) mL, about 1.9(n+1) mL, about 2.0(n+1) mL, about 2.1(n+1) mL, about 2.2(n+1) mL, about 2.3(n+1) mL, about 2.4(n+1) mL, about 2.5(n+1) mL, about 2.6(n+1) mL, about 2.7(n+1) mL, about 2.8(n+1) mL, about 2.9(n+1) mL, about 3.0(n+1) mL, about 3.1(n+1) mL, about 3.2(n+1) mL, about 3.3(n+1) mL, about 3.4(n+1) mL, about 3.5(n+1) mL, about 3.6(n+1) mL, about 3.7(n+1) mL, about 3.8(n+1) mL, about 3.9(n+1) mL, and about 4.0(n+1) mL.

In one embodiment, when a precipitation aid is used, the mixture containing the precipitated oligonucleotide may be subjected to a solid/liquid separation such as filtration. In another embodiment, the precipitated oligonucleotide may be recovered from the precipitation aid by extraction with a polar organic solvent. Suitable solvents include, but are not limited to, carbonyl-type solvents such as acetone, nitrogen-containing solvents such as acetonitrile, and halogenated hydrocarbons such as methylene chloride and chloroform, any combination thereof, and the like.

In yet another embodiment, the oligonucleotide obtained from the above precipitation treatment may be further purified by extraction of the organic solvent with water. This extraction step separates polar impurities, which dissolve in aqueous layer, from the desired oligonucleotide. Exemplary organic solvents include, but are not limited to polar organic solvents such as nitrogen-containing solvents, acetonitrile, formamides, DMF, N-heterocycles, pyridine, carbonyl-type solvents, acetone, THF, DMSO, any combinations thereof, and the like.

In one embodiment, the volume of organic solvent used for the extraction may range from bout 2.0(n+1) mL to 8.0(n+1) mL, where n is the millimoles number of the phosphorothioate triester linkage. In another embodiment, the volume of organic solvent used for the extraction may be about 4.0(n+1) mL. In yet another embodiment, the volume of organic solvent used for the extraction may be about 2.0(n+1) mL, about 2.1(n+1) mL, about 2.2(n+1) mL, about 2.3(n+1) mL, about 2.4(n+1) mL, about 2.5(n+1) mL, about 2.6(n+1) mL, about 2.7(n+1) mL, about 2.8 (n+1) mL, about 2.9(n+1) mL, about 3.0(n+1) mL, about 3.1 (n+1) mL, about 3.2(n+1) mL, about 3.3 (n+1) mL, about 3.4(n+1) mL, about 3.5 (n+1) mL, about 3.6(n+1) mL, about 3.7(n+1) mL, about 3.8(n+1) mL, about 3.9(n+1) mL, about 4.0(n+1) mL, about 4.1 (n+1) mL, about 4.2(n+1) mL, about 4.3 (n+1) mL, about 4.4(n+1) mL, about 4.5 (n+1) mL, about 4.6(n+1) mL, about 4.7(n+1) mL, about 4.8 (n+1) mL, about 4.9(n+1) mL, about 5.0(n+1) mL, about 5.1 (n+1) mL, about 5.2(n+1) mL, about 5.3 (n+1) mL, about 5.4(n+1) mL, about 5.5 (n+1) mL, about 5.6(n+1) mL, about 5.7(n+1) mL, about 5.8 (n+1) mL, about 5.9(n+1) mL, about 6.0(n+1) mL, about 6.1 (n+1) mL, about 6.2(n+1) mL, about 6.3 (n+1) mL, about 6.4(n+1) mL, about 6.5 (n+1) mL, about 6.6(n+1) mL, about 6.7(n+1) mL, about 6.8 (n+1) mL, about 6.9(n+1) mL, about 7.0(n+1) mL, about 7.1 (n+1) mL, about 7.2(n+1) mL, about 7.3 (n+1) mL, about 7.4(n+1) mL, about 7.5 (n+1) mL, about 7.6(n+1) mL, about 7.8(n+1) mL, about 7.9(n+1) mL, and about 8.0(n+1) mL.

In one embodiment, the organic solution containing the desired oligonucleotide may be extracted with an aqueous medium such as water. In one embodiment, the volume of the aqueous medium used for the extraction may be from about 0.5 volume equivalents to about 1.5 volume equivalents relative to the organic solvent. In another embodiment, the volume of the aqueous medium used for the extraction may be about 0.7 volume equivalents relative to the organic solvent. In one embodiment, the volume of the aqueous medium used for the extraction may be about 0.5 volume equivalents, about 0.6 volume equivalents, about 0.7 volume equivalents, about 0.8 volume equivalents, about 0.9 volume equivalents, about 1.0 volume equivalents, about 1.1 volume equivalents, about 1.2 volume equivalents, about 1.3 volume equivalents, about 1.4 volume equivalents, and about 1.5 volume equivalents relative to the organic solvent. After treatment with the aqueous medium, the oligonucleotide-containing layer may be separated by normal techniques and may be further processed, if appropriate, to obtain the desired, purified oligonucleotide.

Deprotection to Yield a Phosphorothioate Oligonucleotide

The oligonucleotides described herein may be deprotected to yield phosphorothioate oligonucleotides. This deprotection may be accomplished by cleaving the S—R₂ bond or bonds of the desired oligonucleotide. In one embodiment, the S—R₂ bond may be cleaved by reaction with a base. Suitable bases include, but are not limited to, alkyl amines, cycloalkyl amines, and aromatic amines. In another embodiment, the base used for cleavage of the S—R₂ bond may be a primary amine, including without limitation a primary straight-chained or branched alkyl amine such as methyl amine, ethyl amine, propyl amine, tert-butyl amine. In yet another embodiment, the base used for cleavage of the S—R₂ bond may be a sterically hindered primary amine.

In still another embodiment, the amine may be a secondary alkyl amine bearing identical or different C₁ to C₈ linear or branched alkyl groups. Suitable secondary alkyl amines include, but are not limited to, dimethyl amine and diethyl amine.

In one embodiment, the cleavage of the S—R₂ bond may be carried out in the presence of any sterically hindered base and any activator. Although sterically hindered bases reduce the likelihood of potential side reactions between the base and the nucleobase moiety, they also generally exhibit decreased reactivity. Accordingly, the activator may be used to produce a clean, fast, and efficient deprotection step. In another embodiment, the sterically hindered base may be tert-butyl amine, aniline, adamantyl amine, or the like. In yet another embodiment, the activator may be an N-heteroaromatic base. In one embodiment, the activator may be a diazole, triazole, tetrazole, derivatives thereof, or any combinations thereof. In still another embodiment, the activator may be 1,2,4-triazole or other triazole or tetrazole derivatives.

In another embodiment, the deprotection of the S-methylene-ester, S-methylene-carbonate, or S-methylene-carbamate group may be accomplished by treating a protected nucleotide with a sterically hindered base such as, and without limitation, tert-butylamine and a diazole, triazole, tetrazole, or any derivative or combination thereof.

In one embodiment, the deprotection may be accomplished by treating a protected nucleotide with a substituted or unsubstituted aromatic amine. In one embodiment, the aromatic amine may be a substituted aniline, wherein the aryl group of the aniline contains linear or branched alkyl or aryl substituents at the 2- and/or 6-positions. Suitable aromatic amines include but are not limited to 2,6-dimethylaniline and 2,6-diethylaniline.

In one embodiment, the deprotection may be accomplished in any desired solvent. In another embodiment, the deprotection may be carried out in polar aprotic solvents. In still another embodiment, the deprotection may be carried out in polar aprotic solvent containing nitrogen. Suitable solvents include, without limitation, N-heterocyclic solvents such as pyridine and the like.

In one embodiment, the deprotection may be carried out at a temperature from about −30° C. to about 70° C. In another embodiment, the deprotection may be carried out at a temperature from about 0° C. to about 30° C. In still another embodiment, the deprotection may be carried out at a temperature of about −30° C., −25° C., −20° C., −15° C., −10° C., −5° C., 0° C., 5° C., 10° C., 15° C., 20° C., 25° C., 30° C., 35° C., 40° C., 45° C., 50° C., 55° C., 60° C., 65° C., or 70° C.

In one embodiment, the liquid reaction medium may contain from about 5% to about 80% by weight of the protected nucleotide relative to the total weight of the reaction mixture. In another embodiment, the liquid reaction medium may contain at least 20% by weight of the protected nucleotide relative to the total weight of the reaction mixture. In still another embodiment, the liquid reaction medium may contain at least 50% by weight of the protected nucleotide relative to the total weight of the reaction mixture. In a further embodiment, the liquid reaction medium may contain about 5% by weight, 10% by weight, 15% by weight, 20% by weight, 25% by weight, 30% by weight, 35% by weight, 40% by weight, 45% by weight, 50% by weight, 55% by weight, 60% by weight, 65% by weight, 70% by weight, 75% by weight, or 80% by weight of the protected oligonucleotide relative to the total weight of the reaction medium.

In another embodiment, the base used for cleavage of the S—R₂ bond of the oligonucleotide may be present in an amount from about 3n mmol to about 20n mmol, wherein n is the number of millimoles of the phosphorothioate trimester linkage. In one embodiment, the base used for cleavage of the S—R₂ bond of the oligonucleotide may be present in an amount of about 3.0n mmol, 3.5n mmol, 4.0n mmol, 4.5n mmol, 5.0n mmol, 5.5n mmol, 6.0n mmol, 6.5n mmol, 7.0n mmol, 7.5n mmol, 8.0n mmol, 8.5n mmol, 9.0n mmol, 10.0n mmol, 10.5n mmol, 11.0n mmol, 11.5n mmol, 12.0n mmol, 12.5n mmol, 13.0n mmol, 13.5n mmol, 14.0n mmol, 14.5n mmol, 15.0n mmol, 15.5n mmol, 16.0n mmol, 16.5n mmol, 17.0n mmol, 17.5n mmol, 18.0n mmol, 18.5n mmol, 19.0n mmol, 19.5n mmol, or 20.0n mmol.

In still another embodiment, the activator used for the cleavage of the S—R₂ bond of the oligonucleotide may be present in an amount from about 0.2n mmol to about 5n mmol, wherein n is the number of millimoles of the phosphorothioate trimester linkage. In a further embodiment, the activator used for the cleavage of the S—R₂ bond of the oligonucleotide may be present in an amount of about 0.2n mmol, 0.3n mmol, 0.4n mmol, 0.5n mmol, 0.6n mmol, 0.7n mmol, 0.8n mmol, 0.9n mmol, 1.0n mmol, 1.1n mmol, 1.2n mmol, 1.3n mmol, 1.4n mmol, 1.5n mmol, 1.6n mmol, 1.7n mmol, 1.8n mmol, 1.9n mmol, 2.0n mmol, 2.1n mmol, 2.2n mmol, 2.3n mmol, 2.4n mmol, 2.5n mmol, 2.6n mmol, 2.7n mmol, 2.8n mmol, 2.9n mmol, 3.0n mmol, 3.1n mmol, 3.2n mmol, 3.3n mmol, 3.4n mmol, 3.5n mmol, 3.6n mmol, 3.7n mmol, 3.8n mmol, 3.9n mmol, 4.0n mmol, 4.1n mmol, 4.2n mmol, 4.3n mmol, 4.4n mmol, 4.5n mmol, 4.6n mmol, 4.7n mmol, 4.8n mmol, 4.9n mmol, 5.0n mmol.

Should the disclosure of any patents, patent applications, and publications which are incorporated herein by reference conflict with the description of the present application to the extent that it may render a term unclear, the present description shall take precedence.

### EXAMPLES

As used herein and throughout this specification, the following abbreviations are defined as follows:

Ap, Gp, and Tp are the 2-deoxyribose nucleobases as previously described and are functional derivatives of A, G, and T nucleobases, respectively. Ap is a 2-deoxyribose nucleobase and is the N-(purin-6-yl)benzamide derivative of adenine. Gp is a 2-deoxyribose nucleobase and is the N-(6-(2,5,-dichlorophenoxy)-purin-2-yl)isobutyramide derivative of guanine. Tp is a nucelobase and is the 5-methyl-4-phenoxypyrimidin-2-one derivative of thymine.

Ap(S—R^{A}), Gp(S—R^{A}), and Tp(S—R^{A}) are the corresponding O═P-thiomethyl propionates (i.e., —SCH₂OC(O)CH₂CH₃) of Ap, Gp, and Tp, respectively. Ap(S—R^{B}), Gp(S—R^{B}), and Tp(S—R^{B}) are the corresponding O═P-thiomethyl isobutyrates (i.e., —SCH₂OC(O)CH(CH₃)₂) of Ap, Gp, and Tp, respectively. Ap(S—R^{c}), Gp(S—R^{c}), and Tp(S—Rc) are the corresponding O═P-chlorothiophenols (i.e., —SC₆H₄Cl) of Ap, Gp, and Tp, respectively. Ap(S—R^{D}), Gp(S—R^{D}), and Tp(S—R^{D}) are the corresponding O═P-nitrothiophenols (i.e., —SC₆H₄NO₂) of Ap, Gp, and Tp, respectively. Ap(H), Gp(H), and Tp(H) are the corresponding O═P—H phosphonates of Ap, Gp, and Tp, respectively. The same nomenclature system is used for the non-derivatized A, G, and T nucleobases. By way of example, A(S—R^{A}) is the corresponding O═P-thiomethyl proprionate of A, and A(H) is the corresponding O═P—H phosphonate of A.

DMTr is the bis-para-methoxytrityl protecting group, which is known to those of ordinary skill in the art, bonded to the 5′-O of the corresponding oligonucleotide as previously described. Lev is the pentan-1,4-dione protecting group, which is also known to those of ordinary skill in the art, bonded to the 3′-O of the corresponding oligonucleotide as previously described.

The convention of these abbreviations used in this disclosure is further illustrated below.

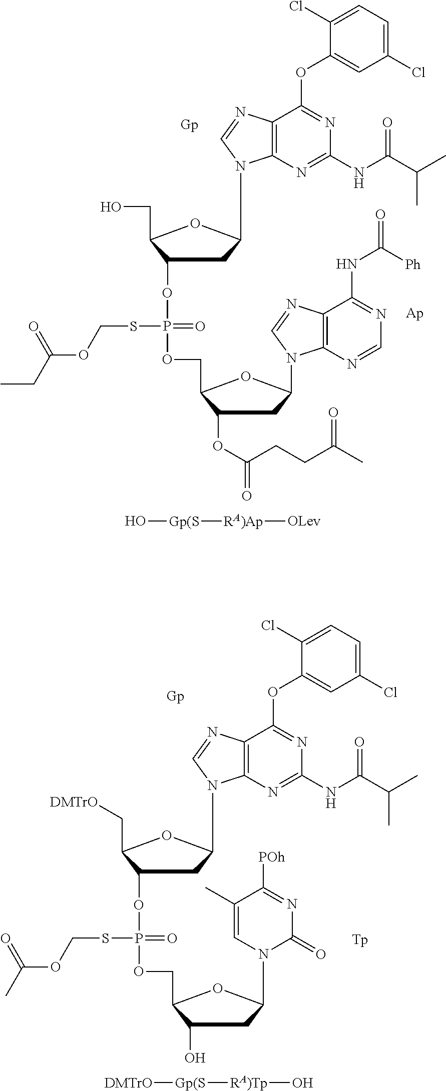

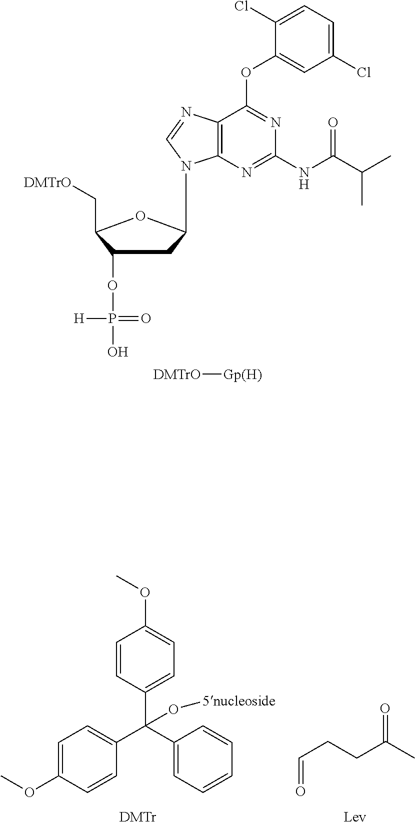

### Example 1

### Synthesis of Propionic Acid Acetylsulfanylmethyl Ester, (CH₃CH₂CO₂CH₂SC(O)CH₃)

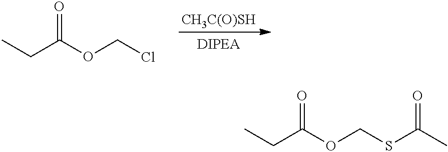

A 2000 mL dry three-necked round bottom flask was equipped with a mechanical stirrer, a dropping funnel, and a N₂ inlet. 168.0 g (1370.9 mmol) of chloromethyl propionate, 1000 mL of anhydrous CH₂Cl₂, and 194.9 g (1508.0 mmol) of diisopropylethylamine were added to the flask. The solution was stirred and cooled in an ice-water bath. 98.0 mL (1370.9 mmol) of thioacetic acid was added slowly added over a period of 30 minutes. After complete addition, the mixture was warmed slowly to room temperature and stirred at room temperature overnight. The solution was concentrated, and a mixture of 500 mL of ethyl acetate and 500 mL of MTBE was added. The resulting mixture was filtered, and the solid was washed with a mixture of 100 mL of ethyl acetate and 100 mL of MTBE. The filtrate was concentrated and the residue was distilled to yield the desired product as a yellow oil. (Yield: 169.8 g, 76.4%)

The corresponding isobutyric acid acetylsulfanylmethyl ester, ((CH₃)₂CHC(O)OCH₂SC(O)CH₃), may be synthesized by a similar procedure.

### Example 2

### Synthesis of propionyloxymethylsulfenyl chloride, (CH₃CH₂C(O)OCH₂SCl)

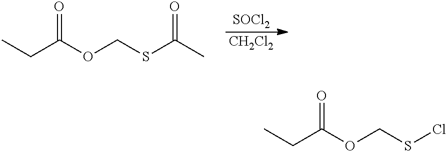

A solution of 9.803 g (60.43 mmol) of propionic aciacetylsulfanylmethyl ester in 80 mL of anhydrous dichloromethane was placed in a 250 mL 3-neck round bottom flask equipped with a magnetic stirrer, thermocouple, nitrogen line and cooling ice bath. The flask's content was chilled to about 0° C., and a total of 11.33 g (83.94 mmol, 1.38 eq.) of sulfuryl dichloride was added to the solution at a rate sufficient to maintain temperature between 0° C. and 5° C. The cooling bath was removed, and the reaction mixture was stirred at room temperature for 1.5 hours. The resulting yellow solution was concentrated to yield 8.88 g of crude methylacetylsulfenyl chloride as a viscous yellow oil.

The corresponding isobutryloxymethylsulfenyl chloride, ((CH₃)CHC(O)OCH₂SC1), may be synthesized by a similar procedure.

### Example 3

### Synthesis of chlorophenylsulfenyl chloride (ClSC₆H₅Cl)

A solution containing 20.15 g (0.139 mol) of 4-chlorothiophenol dissolved in 170 mL of anhydrous carbon tetrachloride was placed in a 0.5 L 3-neck round bottom flask equipped with a magnetic stirrer, thermocouple, addition funnel, nitrogen line, heating mantle, condenser and caustic scrubber to absorb acidic gases (SO₂ and HCl). A catalytic amount of pyridine (3 mL) was added to the solution, and the flask's content was chilled to =10° C. 55.2 g (0.409 mol) sulfuryl chloride was slowly added to the solution, and the reaction mixture was gently refluxed for about 3 hours. The resulting red solution was cooled to room temperature, quickly filtered through glass wool, and concentrated by rotary evaporation to yield 23 g of 4-chlorobenzene sulfenyl chloride as a viscous red oil. This crude material was used immediately for preparation of sulfurized oligonucleotides.

### Example 4

### Synthesis of Solid-Supported N-methyl-sulfonamide 26

163.7 mL (327.5 mmol, 10 eq) of a 2.0 M solution of methylamine dissolved in THF was cooled to 0° C. under argon. 13.46 g (32.75 mmol, 1 eq) of polystyrene-bound sulfonyl chloride 22 (commercially available as “sulfonyl chloride, polymer bound”) was added resulting in a slurry. The slurry was stirred at 0° C. to ambient temperature for overnight. The polystyrene-bound N-methyl-sulfonamide compound was collected by filtration and washed with 50 mL of THF followed by two 200 mL aliquots of dichloromethane. The compound was shaken with 100 mL 0.1 N NaOH, collected by filtration, and then washed with three 100 mL aliquots of deionized water until the washes were neutral, as determined by pH paper. The polystyrene-bound N-methyl-sulfonamide was finally washed with three 100 mL aliquots of acetonitrile, followed by two 75 mL aliquots of dichloromethane, and dried in vacuo to yield 13.2 g of 26 as a pale colored solid.

### Example 5

### Synthesis of Solid-Supported Sulfurization Reagent 13

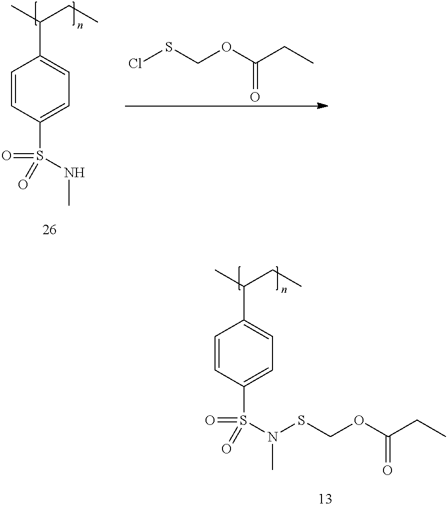

Under an argon atmosphere, a slurry of 1.53 g (3.78 mmol) of polystyrene-bound aminosulfonyl 26 was prepared in 30 mL anhydrous dichloromethane with molecular sieves. 550 μL (6.81 mmol, 1.8 eq) of pyridine was added to the slurry, and the mixture was then cooled in an ice bath. 880 mg (5.68 mmol, 1.5 eq) of methylacetylsulfenyl chloride was slowly added dropwise. The mixture was warmed to room temperature and stirred for about 3 hours. The reaction mixture was then separated from the molecular sieves by decantation, and the resin was separated from the reaction solution by filtration. The resin was washed with 4 aliquots of 75 mL of dichloromethane and dried in vacuo to yield 1.74 g of 13 as a pale-colored solid.

### Example 6

### Synthesis of Solid-Supported Sulfurization Reagent 14

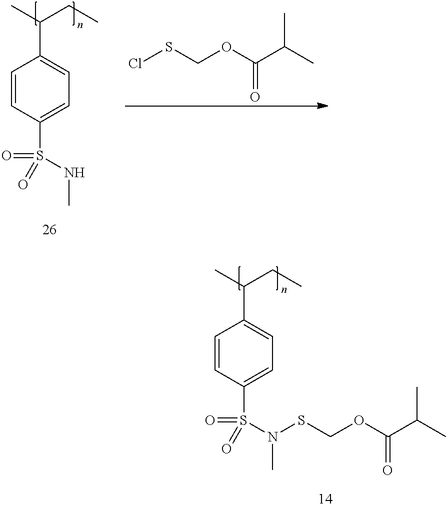

45.69 g of solid-supported sulfurization reagent 26 was placed under an argon atmosphere in a 500 mL three-neck flask equipped with an addition funnel and a mechanical stirrer. 200 mL of anhydrous dichloromethane was transferred to the flask, and the resulting mixture was stirred and cooled to between about 0° C. and 5° C. in an ice bath. 31.9 mL of pyridine and 47.5 g of isobutryloxymethylsulfenyl chloride was added to the mixture, which was then allowed to warm to room temperature and stirred for three hours. The product was filtered and washed with three aliquots of 250 mL of dichloromethane. The product was dried overnight in vacuo to produce 50.7 g of 14 having a load of about 1.15 mmol of the sulfur transfer group per gram of the reagent.

### Example 7

### Synthesis of Solid-Supported Sulfurization Reagent 15

Under an argon atmosphere, a slurry of 1.00 g (2.46 mmol) of polystyrene-bound aminosulfonyl 26 was prepared in about 12 mL of anhydrous dichloromethane with molecular sieves. 360 μL (4.43 mmol mmol, 1.8 eq) of pyridine was added to the slurry, and the mixture is then cooled in an ice bath. 4-chlorophenylsulfenyl chloride from Example 3 (618 mg, 3.45 mmol), 1.8 eq) was subsequently added dropwise to maintain the temperature at about 0° C. The mixture was allowed to warm to room temperature and stirred for 3 hours. The reaction mixture was separated from the molecular sieves by decantation, and the resin was separated from the reaction solution by filtration. The resin is washed with four aliquots of 75 mL of dichloromethane and dried in vacuo to yield 15 as a pale-orange solid.

### Example 8

### Synthesis of Solid-Supported Sulfurization Reagent 16

Under an argon atmosphere, a slurry of 1.00 g (3.70 mmol) of polystyrene-bound aminosulfonyl 26 was prepared in about 16 mL anhydrous dichloroethane with molecular sieves. 360 μL (6.81 mmol, 1.8 eq) of pyridine was added to the slurry, and the mixture is then cooled in an ice bath. Nitrobenzylsulfenyl chloride (701 mg, 3.70 mmol), which is commercially available, was dissolved in 8 ml of dichloroethane and added slowly to the mixture. The mixture was allowed to warm to room temperature and stirred at 60° C. for about 4 hours. The reaction mixture is separated from the molecular sieves by decantation, and the resin is separated from the reaction solution by filtration. The resin is washed with four aliquots of 75 mL of dichloromethane and dried in vacuo to yield 16 as a pale-colored solid.

### Example 9

### Synthesis of ethyl acetylsulfanylmethyl carbonate

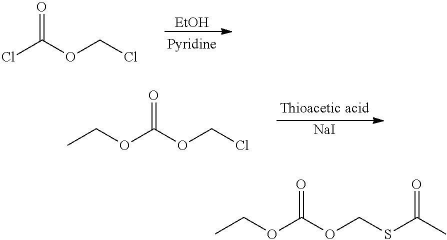

12.9 g (100.0 mmol) of chloromethyl chloroformate is dissolved in 300 mL of anhydrous acetonitrile in a 100 mL dry round bottom flask. The solution is stirred in an ice-water bath, and a mixture of 4.6 g (100.0 mmol) of anhydrous ethanol and 23.7 g of anhydrous pyridine is added slowly over a period of 20 min. After complete addition, the mixture is stirred at room temperature for 1 hour. 1.50 g (10.0 mmol) of sodium iodide is added to the reaction mixture. The mixture is stirred in an ice-water bath, and 7.6 g (100 mmol) thioacetic acid is added over a period of 5 min. After complete addition, the cold bath is removed, and the mixture is stirred at room temperature overnight. 600 mL of hexane is added to the reaction mixture, which is then filtered. The filtrate is concentrated and distilled to yield the desired carbonate compound.

### Example 10

### Synthesis of Solid-Supported Sulfurization Reagent 17

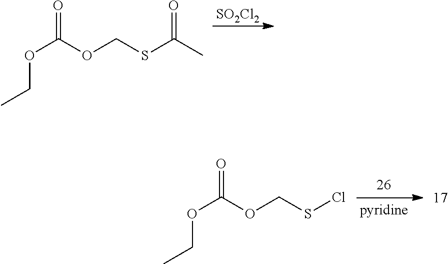

Part A: 8.9 g (50.0 mmol) of ethyl acetylsulfanylmethyl carbonate was placed in a 500 mL dry, round bottom flask containing 200 mL of anhydrous dichloromethane. The solution was cooled in an ice bath under N₂, and 4.0 mL (50.0 mmol) of sulfuryl chloride was slowly added. After complete addition, the mixture was warmed to room temperature and stirred for about 1.5 hours. The solution was evaporated to remove all volatiles. The residue was dissolved in 50 mL of anhydrous dichloromethan to yield Solution A.

Part B: 8.11 g (20 mmol) of solid-supported sulfonamide 26, 3.0 g of 4 Å, activated molecular sieves, and 150 mL of anhydrous dichloromethane are added to a 500 mL dry, round bottom flask. The mixture is cooled in an ice bath under N₂ (g), and 5.3 mL (65.0 mmol) of anhydrous pyridine is added. After complete addition of pyridine, Solution A from Part A is added slowly over 10 minutes. The resulting mixture is then warmed to room temperature and stirred for about 1.5 hours. The reaction mixture is separated from the molecular sieves by decantation, and the resin is separated from the reaction solution by filtration. The resin is washed with four aliquots of 75 mL of dichloromethane and dried in vacuo to yield 17 as a solid.

### Example 11

### Synthesis of acetylsulfanylmethyl dimethylcarbamate

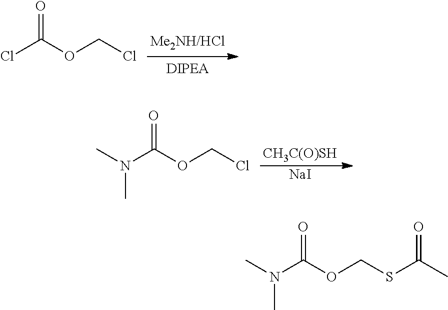

12.9 g (100.0 mmol) of chloromethyl chloroformate and 12.9 g (100 mmol) of dimethylamine hydrochloride is dissolved in 300 mL of anhydrous acetonitrile in a 1000 mL dry, round bottom flask. The mixture is cooled in an bath, and 43.5 mL (250 mmol) of N,N-diisopropylethyl amine is added slowly over 30 min. After complete addition, the mixture is warmed to room temperature and stirred for 1 hour. 1.50 g (10.0 mmol) of sodium iodide is added to the reaction mixture. The mixture is cooled in an ice bath, 7.6 g (100 mmol) of thioacetic acid is added over 5 minutes. After complete addition, the mixture is warmed to room temperature and stirred overnight. 300 mL of hexane is added to the reaction mixture, stirred, and then filtered. The filtrate is concentrated and distilled to give the desired carbamate product.

### Example 12

### Synthesis of Solid-Supported Sulfurization Reagent 18

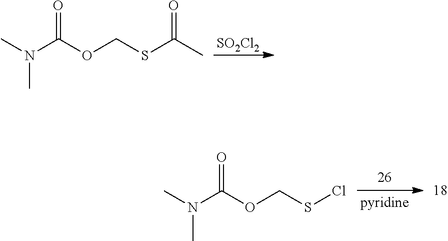

Part A: In a 500 mL dry, round bottom flask, 8.9 g (50.0 mmol) of acetylsulfanylmethyl dimethylcarbamate is placed in 200 mL of anhydrous dichloromethane. The solution is cooled in an ice bath under N₂, and 4.0 mL (50.0 mmol) sulfuryl chloride is slowly added. After addition, the reaction mixture is allowed to warm to room temperature and is stirred for 1.5 hours. The mixture is evaporated to remove all volatiles, and the residue is dissolved in 50 mL of anhydrous dichloromethane to yield Solution A.

Part B: 8.11 g (20 mmol) of solid-supported sulfonamide 26, 3.0 g of 4 Å, activated molecular sieves, and 150 mL of anhydrous dichloromethane are added to a 500 mL dry, round bottom flask. The mixture is cooled in an ice bath under N₂, and 5.3 mL (65.0 mmol) of anhydrous pyridine is added. After complete addition of pyridine, Solution A is added slowly over 10 minutes. The resulting mixture is then allowed to warm to room temperature and stirred for about 1.5 hours. The reaction mixture is separated from the molecular sieves by decantation, and the resin is separated from the reaction solution by filtration. The resin is washed with four aliquots of 75 mL of dichloromethane and dried in vacuo to yield 18 as a solid.

### Example 13

### Comparison of Analytical Control Using Solid-Supported Sulfurization Reagent 13 and its Corresponding Soluble Sulfurization Reagent

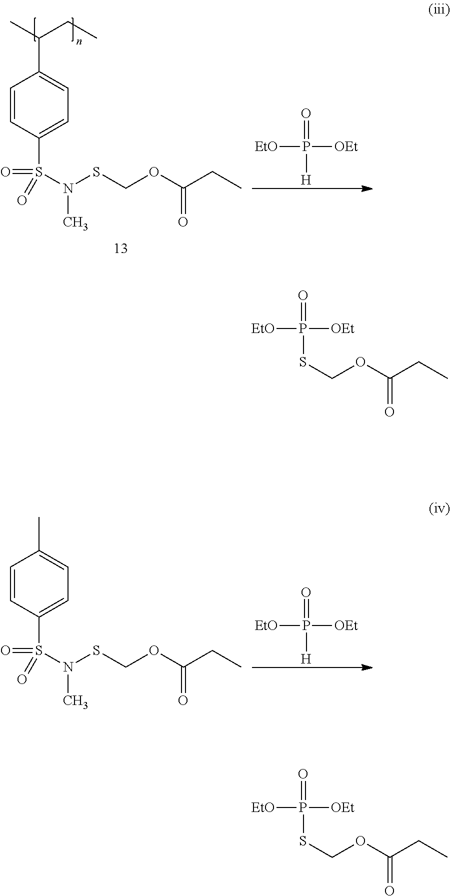

In order to assess the viability of the use of the solid-supported sulfurization reagents described herein in oligonucleotide synthesis, solid-supported sulfurization reagent 13, having a load of 0.67 mmol/g by ³¹P-NMR and 1.0 mmol/g by elemental analysis, was reacted with P(O)(OCH₂CH₃)₂H to yield P(O)(OCH₂CH₃)₂(SCH₂OC(O)CH₂CH₃), as shown in Equation (iii) above. The corresponding soluble sulfurization reagent was also reacted with P(O)(OCH₂CH₃)₂H to yield P(O)(OCH₂CH₃)₂(SCH₂OC(O)CH₂CH₃), as shown in Equation (iv) above. A comparison of the ³¹P-NMR spectra confirmed that the solid-supported sulfurization reagent showed reactivity towards the P—H bond similar to that of the soluble sulfurization reagent. This demonstrates that the solid-supported sulfurization reagent, which was purified by filtration and washing, is a viable replacement for the corresponding soluble sulfurization reagent, which requires purification by chromatography immediately prior to use.

### Example 14

### Synthesis of DMTrO-T(S—R^{A})T-OLev 30 with Isolation of the Intermediate H-phosphonate

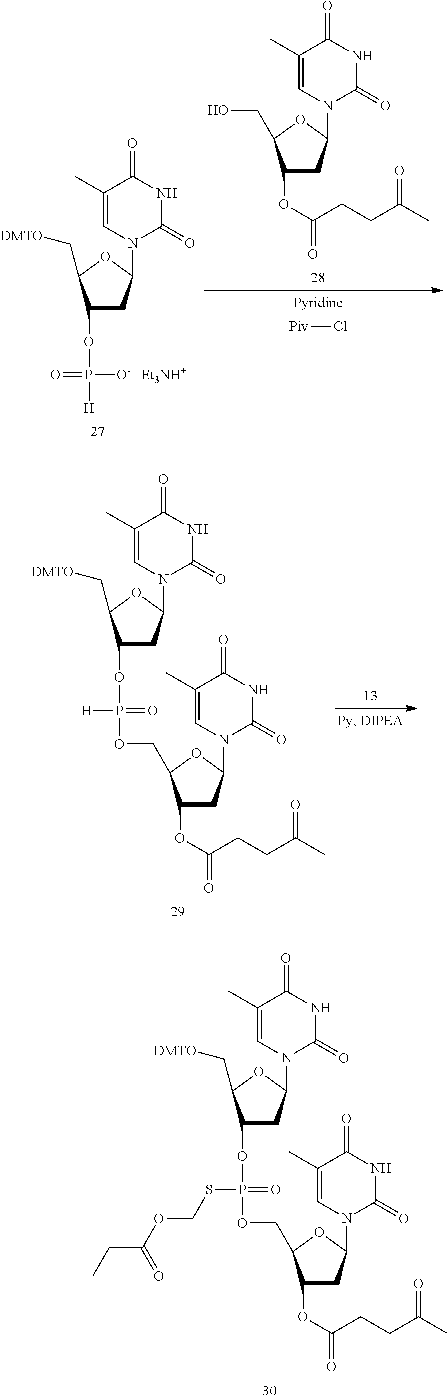

A mixture of 1.86 g (2.62 mmol, 1.15 eq.) of H-phosphonate 27 and 0.78 g (2.28 mmol) of 3′-protected deoxy-thymidine 28 was co-evaporated with anhydrous pyridine (3×25 mL). The oily residue was dissolved in 10 mL of anhydrous pyridine and cooled to about 0° C. under argon atmosphere. A total of 0.56 g (4.66 mmol, 2 eq.) of pivaloyl chloride was added dropwise via syringe, and the resulting mixture was allowed to warm up to ambient temperature. The reaction mixture was stirred for an additional 15 min and then was quenched with 50 g of ice and 100 mL of diluted saturated NaHCO₃ (80 mL water and 20 mL of sodium bicarbonate). The organic material was extracted with dichloromethane (2×80 mL), and the extract was washed with a mixture of cold water (70 mL), saturated sodium bicarbonate (30 mL), and brine (10 mL). The organic layer was dried over anhydrous sodium sulfate and concentrated on a rotavap to yield 6.74 g of crude 29 as clear oil. ³¹P (162 MHz, CDCl₃, δ): 8.58 (s) and 7.06 (s).

A solution of crude H-phosphonate 29 (6.74 g) in 20 mL of anhydrous pyridine was cooled to about 0° C. under argon atmosphere. 2.3 molar equivalents of sulfurization reagent 13 was added to the reaction mixture, and after 5 min, 0.502 g (3.88 mmol, 1.7 eq.) of diisopropylethyl amine was also added to the flask. The reaction was warmed to room temperature and, after an additional hour of stirring, was quenched with a diluted, cold solution of sodium bicarbonate (100 mL). The resin was removed from the reaction mixture by filtration. The organic products were extracted with dichloromethane (2×80 mL), washed with water (100 mL) and dried over sodium sulfate. The organic phase was concentrated on rotavap to yield crude 30 as yellow oil. ³¹P (162 MHz, CDCl₃, δ): 26.77 (s) and 26.67 (s).

### Example 15

### Assay of Solid-Supported Sulfurization Reagent 13 by NMR

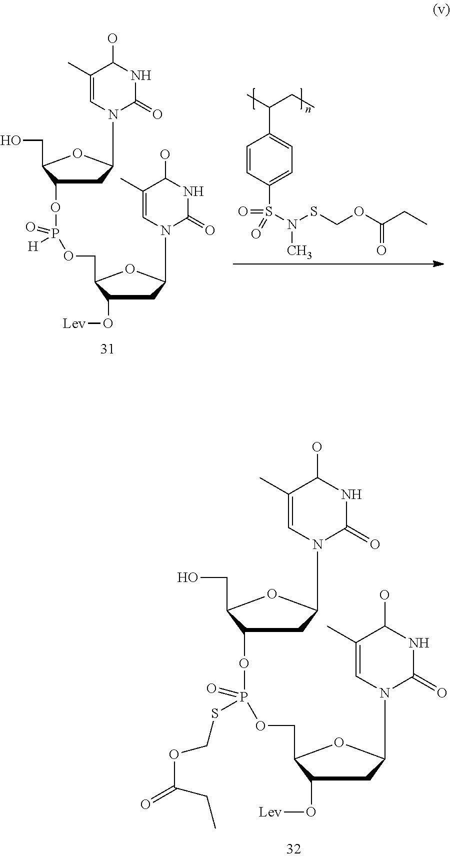

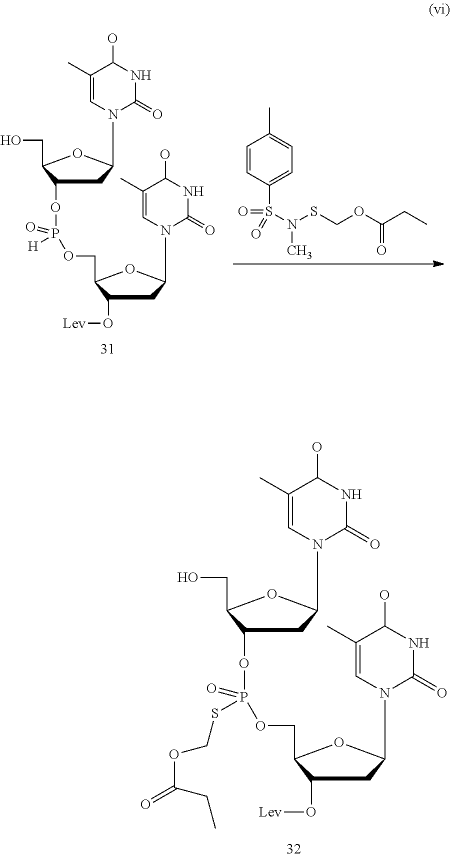

To assess the viability of the use of the solid-supported sulfurization reagents described herein in oligonucleotide synthesis, solid-supported sulfurization reagent 13 was reacted in an NMR tube with the H-phosphonate dinucleotide HO-T(H)T-OLev 31 to yield the sulfurized dinculeotide HO-T(S—R^{A})T-OLev 30, as shown in Equation (v). The corresponding soluble sulfurization reagent was also reacted in an NMR tube with the H-phosphonate dinucleotide HO-T(H)T-OLev 31 to yield the sulfurized dicnucleotide HO-T(S—R^{A})T-OLev 32, as shown in Equation (vi).

A comparison of the ³¹P-NMR spectra confirmed that the solid-supported sulfurization reagent showed reactivity towards the P—H bond of the dinucleotide similar to that of the soluble sulfurization reagent. While the soluble sulfurization reagent required purification by chromatography immediately prior to use, the solid-supported sulfurization reagent was purified by filtration and washing.

### Example 16

### Regeneration of Solid-Supported Sulfonamide Reagent 34

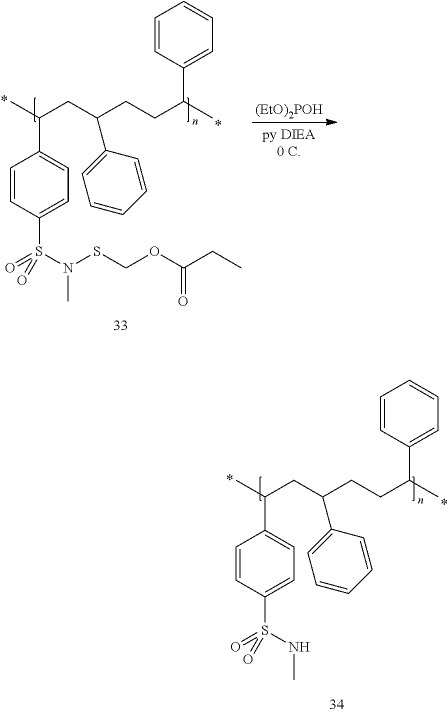

To a slurry of polystyrene derivative 33 (1.05 g, 882.0 mmol) in pyridine (anhydrous, 15 mL) cooled in ice bath was added diethylphosphite (454 μL, 3528.0 mmol, 4 equiv) and then diisopropylethylamine (384 μL, 2205.0 mmol, 2.5 equiv). The cooling bath was removed and the slurry was stirred at ambient temperature for 7 hours. The resin slurry was diluted two-fold with dichloromethane and the product was isolated on a glass frit and washed extensively with dichloromethane (4×40 mL). The product was then placed in vacuo overnight to give 985 mg of 34 as a pale colored solid. The polymer was submitted for CHNS analysis and the sulfur loading of the polymer was determined to be zero by elemental analysis and by ³¹P nmr.

### Example 17

### Synthesis of DMTrO-Ap(S—R^{A})T-OLev

A mixture of triethylammonium 6-N-(benzoyl)-5′-O-(4,4′-dimethoxytrityl)-2′-deoxyadenosine-3′H-phosphonate (4.94 g, 6.0 mmol) and 3′-O-lev ulinylthymidine (1.70 g, 5.0 mmol) is rendered anhydrous by evaporation with pyridine, diluted with anhydrous pyridine (12.5 mL) and stirred under N₂ at 0° C. Subsequently, pivaloyl chloride (1.24 mL, 10.0 mmol) is added slowly over 2 min. The reaction mixture is stirred at 0° C. for 5 min and partitioned between methylene chloride (100 mL) and 1.25 N sodium acetate-acetic acid buffer (2×100 mL). The buffer is made by mixing 190 mL of 1.25 N aqueous sodium acetate solution with 10 mL of 1.25 N aqueous acetic acid solution. The organic layer is dried (Na₂SO₄) and concentrated. The residue is co-evaporated with 50 mL of toluene, dissolved in anhydrous methylene chloride (25 mL), and treated under N₂ (g) at 0° C. with a slurry of 1.5 molar equivalents of solid-supported sulfurization reagent 13 in anhydrous methylene chloride, followed by the addition of N,N-diisopropylethylamine (0.87 mL, 5.0 mmol). After stirring at ambient temperature for 30 min, solution A (MTBE: Hexane=1:2, 37.5 mL) is added over 10 min. The mixture is stirred, and the additional portion of A (37.5 mL) is added over 30 min. The stirring is continued for further 30 min, and the mixture is filtered. The solid is washed with a mixture of solvent made of the solution A and CH₂Cl₂ in the ratio of 5:1 (60 mL). The solid is then extracted with methylene chloride (4×40 mL). The methylene chloride filtrate is concentrated. The residue is dissolved in acetonitrile (20 mL) and stirred in an ice-water bath, and cold water (14 mL) is added over 20 min. The bottom layer is partitioned between methylene chloride (100 mL) and an aqueous (1:1) brine solution (60 mL). The organic layer is dried (Na₂SO₄) and concentrated to yield DMTrO-Ap(S—R^{A})T-OLev as white solid. ³¹P NMR (CDCl₃, 121.5 MHz): δ=26.7, 26.3.

### Example 18

### Synthesis of DMTrO-Ap(S—R^{B})T-OLev

A mixture of triethylammonium 6-N-(benzoyl)-5′-O-(4,4′-dimethoxytrityl)-2′-deoxyadenosine-3′H-phosphonate (4.94 g, 6.0 mmol) and 3′-O-levulinylthymidine (1.70 g, 5.0 mmol) is rendered anhydrous by evaporation with pyridine, diluted with anhydrous pyridine (12.5 mL) and stirred under N₂ at 0° C. Subsequently, pivaloyl chloride (1.24 mL, 10.0 mmol) is added slowly over 2 min. The reaction mixture is stirred at 0° C. for 5 min and partitioned between methylene chloride (100 mL) and 1.25 N sodium acetate-acetic acid buffer (2×100 mL). The buffer is made by mixing 190 mL of 1.25 N aqueous sodium acetate solution with 10 mL of 1.25 N aqueous acetic acid solution. The organic layer is dried (Na₂SO₄) and concentrated. The residue is co-evaporated with 50 mL of toluene, dissolved in anhydrous methylene chloride (25 mL), and treated under N₂ (g) at 0° C. with a slurry of 1.5 molar equivalents of solid-supported sulfurization reagent 14 in anhydrous methylene chloride, followed by the addition of N,N-diisopropylethylamine (0.87 mL, 5.0 mmol). After stirring at ambient temperature for 30 min, solution A (MTBE: Hexane=1:2, 37.5 mL) is added over 10 min. The mixture is stirred, and the additional portion of A (37.5 mL) is added over 30 min. The stirring is continued for further 30 min, and the mixture is filtered. The solid is washed with a mixture of solvent made of the solution A and CH₂Cl₂ in the ratio of 5:1 (60 mL). The solid is then extracted with methylene chloride (4×40 mL). The methylene chloride filtrate is concentrated. The residue is dissolved in acetonitrile (20 mL) and stirred in an ice-water bath, and cold water (14 mL) is added over 20 min. The bottom layer is partitioned between methylene chloride (100 mL) and an aqueous (1:1) brine solution (60 mL). The organic layer is dried (Na₂SO₄) and concentrated to yield DMTrO-Ap(S—R^{B})T-OLev.

### Example 19

### Synthesis of DMTrO-Ap(S—R^{C})T-OLev

A mixture of triethylammonium 6-N-(benzoyl)-5′-O-(4,4′-dimethoxytrityl)-2′-deoxyadenosine-3′H-phosphonate (4.94 g, 6.0 mmol) and 3′-O-levulinylthymidine (1.70 g, 5.0 mmol) is rendered anhydrous by evaporation with pyridine, diluted with anhydrous pyridine (12.5 mL) and stirred under N₂ at 0° C. Subsequently, pivaloyl chloride (1.24 mL, 10.0 mmol) is added slowly over 2 min. The reaction mixture is stirred at 0° C. for 5 min and partitioned between methylene chloride (100 mL) and 1.25 N sodium acetate-acetic acid buffer (2×100 mL). The buffer is made by mixing 190 mL of 1.25 N aqueous sodium acetate solution with 10 mL of 1.25 N aqueous acetic acid solution. The organic layer is dried (Na₂SO₄) and concentrated. The residue is co-evaporated with 50 mL of toluene, dissolved in anhydrous methylene chloride (25 mL), and treated under N₂ (g) at 0° C. with a slurry of 1.5 molar equivalents of solid-supported sulfurization reagent 15 in anhydrous methylene chloride, followed by the addition of N,N-diisopropylethylamine (0.87 mL, 5.0 mmol). After stirring at ambient temperature for 30 min, solution A (MTBE: Hexane=1:2, 37.5 mL) is added over 10 min. The mixture is stirred, and the additional portion of A (37.5 mL) is added over 30 min. The stirring is continued for further 30 min, and the mixture is filtered. The solid is washed with a mixture of solvent made of the solution A and CH₂Cl₂ in the ratio of 5:1 (60 mL). The solid is then extracted with methylene chloride (4×40 mL). The methylene chloride filtrate is concentrated. The residue is dissolved in acetonitrile (20 mL) and stirred in an ice-water bath, and cold water (14 mL) is added over 20 min. The bottom layer is partitioned between methylene chloride (100 mL) and an aqueous (1:1) brine solution (60 mL). The organic layer is dried (Na₂SO₄) and concentrated to yield DMTrO-Ap(S—R^{C})T-OLev.

### Example 20

### Synthesis of DMTrO-Ap(S—R^{D})T-OLev

A mixture of triethylammonium 6-N-(benzoyl)-5′-O-(4,4′-dimethoxytrityl)-2′-deoxyadenosine-3′H-phosphonate (4.94 g, 6.0 mmol) and 3′-O-levulinylthymidine (1.70 g, 5.0 mmol) is rendered anhydrous by evaporation with pyridine, diluted with anhydrous pyridine (12.5 mL) and stirred under N₂ at 0° C. Subsequently, pivaloyl chloride (1.24 mL, 10.0 mmol) is added slowly over 2 min. The reaction mixture is stirred at 0° C. for 5 min and partitioned between methylene chloride (100 mL) and 1.25 N sodium acetate-acetic acid buffer (2×100 mL). The buffer is made by mixing 190 mL of 1.25 N aqueous sodium acetate solution with 10 mL of 1.25 N aqueous acetic acid solution. The organic layer is dried (Na₂SO₄) and concentrated. The residue is co-evaporated with 50 mL of toluene, dissolved in anhydrous methylene chloride (25 mL), and treated under N₂ (g) at 0° C. with a slurry of 1.5 molar equivalents of solid-supported sulfurization reagent 16 in anhydrous methylene chloride, followed by the addition of N,N-diisopropylethylamine (0.87 mL, 5.0 mmol). After stirring at ambient temperature for 30 min, the resin is removed from the reaction mixture by filtration. Solution A (MTBE: Hexane=1:2, 37.5 mL) is added over 10 min. The mixture is stirred, and the additional portion of A (37.5 mL) is added over 30 min. The stirring is continued for further 30 min, and the mixture is filtered. The solid is washed with a mixture of solvent made of the solution A and CH₂Cl₂ in the ratio of 5:1 (60 mL). The solid is then extracted with methylene chloride (4×40 mL). The methylene chloride filtrate is concentrated. The residue is dissolved in acetonitrile (20 mL) and stirred in an ice-water bath, and cold water (14 mL) is added over 20 min. The bottom layer is partitioned between methylene chloride (100 mL) and an aqueous (1:1) brine solution (60 mL). The organic layer is dried (Na₂SO₄) and concentrated to yield DMTrO-Ap(S—R^{D})T-OLev.

### Example 21

### Preparation of DMTrO-Gp(H)

To phosphorous acid (78.7 g, 960.0 mmol) which was made anhydrous by evaporation with pyridine (500 mL), 2′-deoxy-6-O-(2,5-dichlorophenyl)-5′-O-(4,4′-dimethoxytrityl)-2-N-isobutyrylguanosine (62.8 g, 80.0 mmol) was added and the mixture was again rendered anhydrous by evaporation with pyridine. The residue was treated with anhydrous pyridine (480 mL) and pivaloyl chloride (64.0 mL, 520.0 mmol) which was added over 30 min at 10° C. The reaction mixture was stirred for 6 hours at room temperature and concentrated. The residue was dissolved in 800 mL of methylene chloride and washed sequentially with cold water (800 mL), triethylammonium hydrogen carbonate (2.0 N, 400 mL×2). The organic layer was dried (anhydrous Na₂SO₄) and concentrated. The residue was dissolved in 80 mL of methylene chloride, and a solution A (MTBE:Hexane=1:2, 360 mL) was added over 20 min under stirring followed by Celite (80 g). Subsequently, additional solution A (360 mL) was added slowly over 30 min. The mixture was filtered and the solid was washed with MTBE (300 mL). The solid was then extracted with methylene chloride (200 mL×4). The methylene chloride filtrate was concentrated to yield the desired product (70.4 g) as white foam. Yield: 93%. ³¹P NMR (CDCl₃, 121.5 MHz): δ=2.71.

### Example 22

### Preparation of DMTrO-Gp(S—R^{A})Tp-OH

A mixture of triethylammonium 2′-deoxy-6-O-(2,5-dichlorophenyl)-5′-O-(4,4′-dimethoxytrityl)-2-N-isobutyrylguanosine-3′H-phosphonate (54.9 g, 58.0 mmol) and 3′-O-levulinyl-4-O-phenylthymidine (20.1 g, 48.3 mmol) is rendered anhydrous by evaporation with pyridine and diluted with anhydrous pyridine (121 mL). This solution is treated under N₂ at 0° C. with pivaloyl chloride (11.8 mL, 96.6 mmol) over 5 min.

After stirring for additional 5 min, a slurry of solid-suppored sulfurization reagent 13 containing 1.5 molar equivalents of the sulfur transfer group in anhydrous methylene chloride was added, followed by N,N-diisopropylethylamine (8.4 mL, 48.3 mmol). The reaction mixture is allowed to stir at ambient temperature for 30 min and is diluted with 600 mL of methylene chloride. The resin is removed from the reaction mixture by filtration.

The organic layer is washed sequentially with cold water (600 mL), and saturated sodium bicarbonate (500 mL×2), dried (anhydrous Na₂SO₄) and concentrated. The residue is dissolved in 97 mL of methylene chloride, and a solution A (MTBE:Hexane=1:2, 194 mL) is added over 20 min under stirring followed by celite (73 g) and the additional portion of the solution A (194 mL), which is added over 30 min. After stirring for an additional 30 min, the mixture is filtered. The solid is washed with MTBE:Hexane=4:1 (200 mL and was extracted with methylene chloride (150 mL×4). The methylene chloride filtrate is concentrated to yield D product (68.1 g) as yellow foam ³¹P NMR (CDCl₃, 121.5 MHz): δ=26.9, 26.2. This product was used for next step without further purification. To a stirred solution of the above product (64 g) in 117 mL of methylene chloride at 0° C., a cold mixture of pyridine: acetic acid: hydrazine monohydrate=37.5 mL: 25.0 mL: 2.5 mL (51.6 mmol) was added. After stirring at 0° C. for 1 hour, the reaction mixture was diluted with methylene chloride (200 mL) and was washed with cold water (500 mL). The aqueous layer was back extracted with methylene chloride (100 mL). The combined methylene chloride extracts were dried (anhydrous Na₂SO₄) and concentrated. The residue was dissolved in 235 mL of acetonitrile, stirred in an ice-water bath, and treated with cold water (188 mL) which was added gradually over 30 min. The bottom organic layer was separated, diluted with 200 mL of methylene chloride and dried (anhydrous Na₂SO₄). After concentration, the residue was dissolved in 94 mL of methylene chloride and treated sequentially under stirring by solution A (MTBE: Hexane=1:2, 94 mL) added over 20 min, Celite (70 g) and again by the solution A (94 mL) added over 30 min. The mixture was filtered, the solid was washed with MTBE: Hexane=4:1 (300 mL) and extracted with methylene chloride (150 mL×4). The methylene chloride filtrate was concentrated to give the product (52 g) as yellow foam. Yield: 87%. This product was used for next step directly. ³¹P NMR (CDCl₃, 121.5 MHz): δ=28.1, 25.3.

### Example 23

### Complete Deprotection of HO-Gp(S—R^{A})Tp(S—R^{A})Gp(S—R^{A})A-OLey, a 5′-OH Fully Protected Oligonucleotide Phosphorothioate

Fully-protected tetramer HO-Gp(S—R^{A})Tp(S—R^{A})Gp(S—R^{A})A-OLev (1.38 g, 0.62 mmol) was rendered anhydrous by evaporation of added pyridine. 192.7 mg (2.79 mmol) of 1,2,4-triazole, 1.5 g of 4 Å molecular sieve, and 6.0 mL of anhydrous pyridine were added to the residue. This mixture was stirred and cooled to about 0° C. under N₂, and 1.95 mL (18.6 mmol) of tert-butylamine were added. The resulting mixture was then warmed to room temperature and stirred for 4 hours. The mixture was filtered and the molecular sieve was washed with two aliquots of 5 mL of pyridine. The combined filtrate was concentrated to dryness. To the residue, syn-2-pyridinealdoxime (909 mg, 7.44 mmol) was added, followed by anhydrous acetonitrile (10 mL). The mixture was stirred and cooled to about 0° C., and 1,8-diazabicyclo[5.4.0]undec-7-ene (1.67 mL, 11.2 mmol) was added. After stirring at room temperature for 15 hours, MTBE (50 mL) was added slowly over 10 min. After stirring further for 20 min, the top clear solution was decanted, and the residue was rinsed with ethyl acetate (20 mL). The residue was evaporated to remove the residual solvents and was dissolved in a mixture of 28% aqueous ammonia (10.0 mL) and 2-mercaptoethanol (0.5 mL). The resulting mixture was heated at about 55° C. for 15 hours. After cooling, the mixture was added dropwise to a stirring mixture of isopropanol: THF=1:3 (80 mL) over 10 min. After stirring further for 20 min, the top clear solution was decanted, and the residue was rinsed with THF (20 mL). The residue was purified with a reversal C18 chromatography. The product obtained was applied to a column (8 cm×3 cm diameter) of Amberlite IR-120 (plus) ion-exchange resin (sodium form). The column was eluted with water, and the desired fractions were combined and lyophilized to give the sodium form of the fully-deprotected oligonucleotide phosphorothioate of product 684 mg as white solid. Yield: 83%. ³¹P NMR (D₂O, 121.5 MHz): δ=55.4-54.6 (m).

### Example 24

### Synthesis of a DMTrO-T(S—R^{A})T-OLev Without Isolation of the Intermediate H-phosphonate

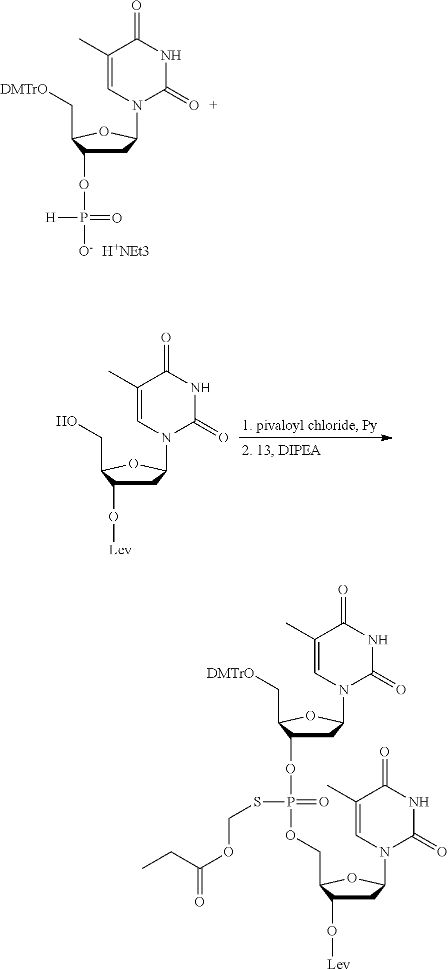

A solution of triethylammonium 5′-O-(4,4′-dimethoxyftityl)-thymidine-3′-H-phosphonate (425.9 mg, 0.6 mmol), 3′-O-levulinyl thymidine (170.2 mg, 0.5 mmol) and dry pyridine (10.0 mL) is rotary evaporated to dryness. The residue is redissolved in 10 mL of pyridine and rotary evaporated again to dryness. To the residue, molecular sieves (300 mg, activated, 3 Å) and anhydrous pyridine (5.0 mL) were added. The mixture is stirred at room temperature under N₂, pivaloyl chloride (0.22 mL, 1.75 mmol) was added. After stirring at room temperature for 5 min, a slurry containing 1.5 molar equivalents of solid-supported sulfurization reagent 13 in CH₂Cl₂ was added, followed by DIPEA (0.17 mL, 2.0 mmol). The resulting mixture is stirred at room temperature for 30 min. The resin is filtered by filtration. Ethyl acetate (30 mL) is added. The reaction solution is then filtered and the filtrate is washed with water (15 mL), semi-saturated aqueous sodium bicarbonate (15 mL×2) and brine (15 mL). The organic layer is dried and evaporated to yield a pale yellow oil. This crude oil is purified by silica gel chromatography (EtOAc/Acetone) to give yield DMTrO-T(S—R^{A})T-OLev as colorless foam.

### Example 25

### Preparation of DMTrO-T(S—R⁵T-OLev

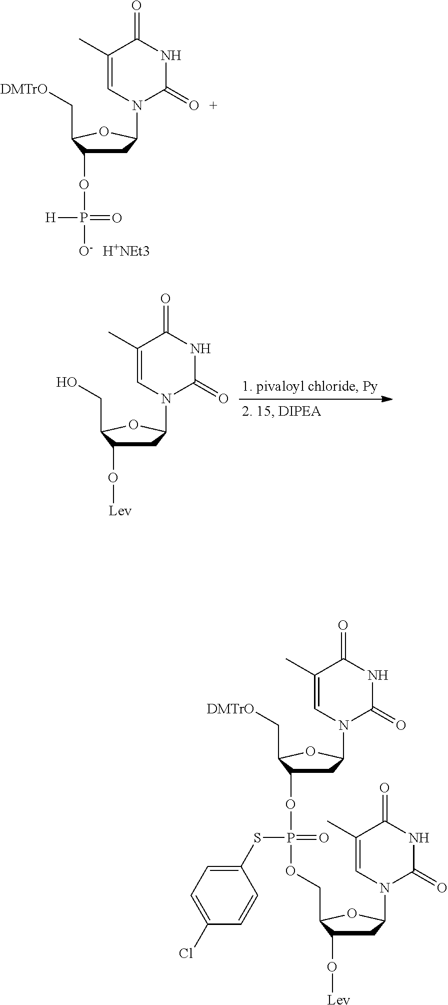

A solution of triethylammonium 5′-dimethoxytrityl)-thymidine-3′-H-phosphonate (4.26 g, 6.0 mmol) and 3′-O-levulinylthymidine (1.70 g, 5.0 mmol) in 60 mL of pyridine is evaporated to dryness. To the residue, anhydrous pyridine (12.5 mL) is added and the resulting solution is stirred under argon at 0° C. Pivaloyl chloride (1.24 mL, 10.0 mmol) is slowly added over 3 minutes. The mixture is stirred further at 0° C. for 5 minutes.

A solution of 6 mmol of solid-supported sulfurizing agent 15 in anhydrous dichloromethane or anhydrous pyridine is added, followed by N,N-diisopropylethylamine (0.87 mL, 5.0 mmol) over 2 minutes. The cold bath is removed, and the mixture is stirred at ambient temperature for 30 minutes. The reaction mixture is diluted with 100 mL of dichloromethane and is washed with cold water (100 mL), followed by saturated sodium bicarbonate (100 mL). The resin is removed by filtration. The organic layer is dried (Na₂SO₄) and concentrated. The residue is dissolved in 10 mL of dichloromethane and 0.1 mL of triethylamine, and 16 mL of Solution A containing MTBE and hexane (2:1) is added slowly over 15 minutes under stirring followed by celite (7.5 g). The mixture is stirred and an additional 16 mL of Solution A is added slowly over 15 minutes. After further stirring for 30 minutes, the mixture is filtered. The solid is washed with a mixture of CH₂Cl₂/Solution A, 1:4 v/v (50 mL×2). The solid is then washed with dichloromethane (50 mL×3). The dichloromethane filtrate is concentrated. The residue is dissolved in 40 mL of acetonitrile and stirred in an ice-water bath, and then water (36 mL) is added over 20 minutes. The bottom organic layer is separated and partitioned between dichloromethane (80 mL) and a mixture of saturated sodium bicarbonate (30 mL) and brine (30 mL). The organic layer is dried (Na₂SO₄) and concentrated to yield DMTrO-T(S—R^{C})T-OLev.

### Example 26

### Complete Deprotection of a HO-Gp(S—R^{A})T(S—R^{C}T-OLev, a Mixed 5′-OH Fully Protected Trimeric Oligonucleotide

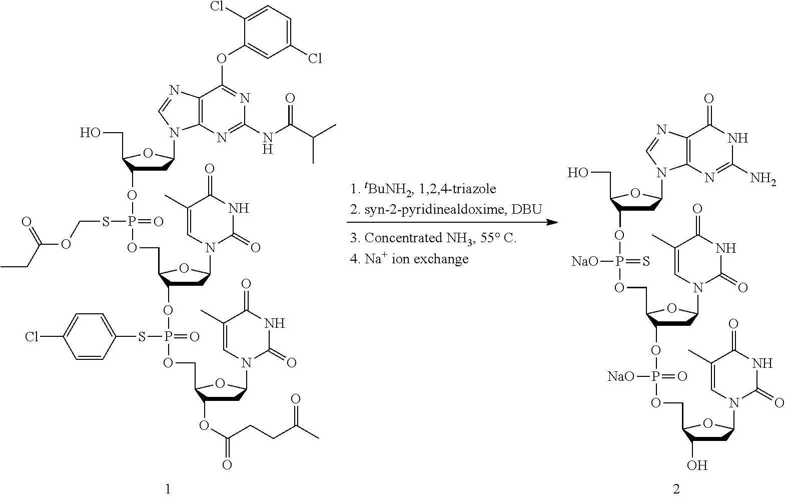

Fully-protected trimer (7.09 g, 5.0 mmol) is rendered anhydrous by evaporation of added pyridine. To the residue, 1,2,4-triazole (690.7 mg, 10.0 mmol), 4 Å molecular sieve (2.0 g) and anhydrous pyridine (25.0 mL) are added. This mixture is stirred and cooled to 0° C. under N₂, and tert-butylamine (2.19 g, 30.0 mmol) is added. The resulting mixture then is stirred at room temperature for 4 hours. To the solution, syn-2-pyridinealdoxime (2.44 g, 20.0 mmol) is added, followed by 1, 8-diazabicyclo[5.4.0]undec-7-ene (6.09 g, 40.0 mmol). After stirring at room temperature for 15 hours, the molecular sieve is filtered and washed with pyridine (10.0 mL). The filtrate is concentrated. The residue is dissolved in 28% aqueous ammonia (25.0 mL). The resulting solution is heated at 55° C. for 15 hours. After cooling down the mixture is concentrated and purified with a reversal C18 chromatography. The product obtained is applied to a column (100 g) of Amberlite® IR-120 (plus) ion-exchange resin (sodium form). The column is eluted with water, and the desired fractions are combined and lyophilized to give the desired product 2.

### Example 27

### Preparation of DMTrO-T(S—R^{D})T-OLev

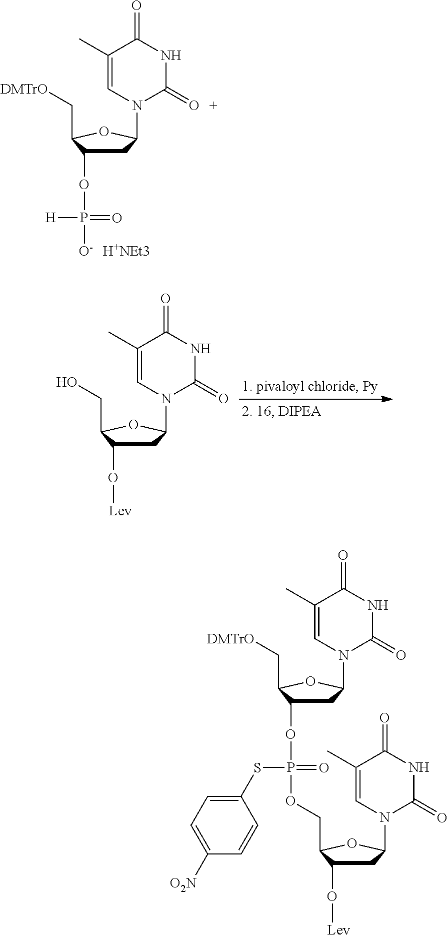

A solution of triethylammonium 5′-O-(4,4′-dimethoxytrityl)-thymidine-3′-H-phosphonate (4.26 g, 6.0 mmol) and 3′-O-levulinylthymidine (1.70 g, 5.0 mmol) in 60 mL of pyridine is evaporated to dryness. To the residue, anhydrous pyridine (12.5 mL) is added and the resulting solution is stirred under argon at 0° C. Pivaloyl chloride (1.24 mL, 10.0 mmol) is slowly added over 3 minutes. The mixture is stirred further at 0° C. for 5 minutes.

A solution of 6 mmol of solid-supported sulfurizing agent 16 in anhydrous dichloromethane or anhydrous pyridine is added, followed by N,N-diisopropylethylamine (0.87 mL, 5.0 mmol) over 2 minutes. The cold bath is removed, and the mixture is stirred at ambient temperature for 30 minutes. The reaction mixture is diluted with 100 mL of dichloromethane and is washed with cold water (100 mL), followed by saturated sodium bicarbonate (100 mL). The resin is removed by filtration. The organic layer is dried (Na₂SO₄) and concentrated. The residue is dissolved in 10 mL of dichloromethane and 0.1 mL of triethylamine, and 16 mL of Solution A containing MTBE and hexane (2:1) is added slowly over 15 minutes under stirring followed by celite (7.5 g). The mixture is stirred and an additional 16 mL of Solution A is added slowly over 15 minutes. After further stirring for 30 minutes, the mixture is filtered. The solid is washed with a mixture of CH₂Cl₂/Solution A, 1:4 v/v (50 mL×2). The solid is then washed with dichloromethane (50 mL×3). The dichloromethane filtrate is concentrated. The residue is dissolved in 40 mL of acetonitrile and stirred in an ice-water bath, and then water (36 mL) is added over 20 minutes. The bottom organic layer is separated and partitioned between dichloromethane (80 mL) and a mixture of saturated sodium bicarbonate (30 mL) and brine (30 mL). The organic layer is dried (Na₂SO₄) and concentrated to yield DMTrO-T(S—R^{D})T-OLev.

### Example 28

### Preparation of DMTrO-T(S—R^{B})T(H)—OH.NEt₃39

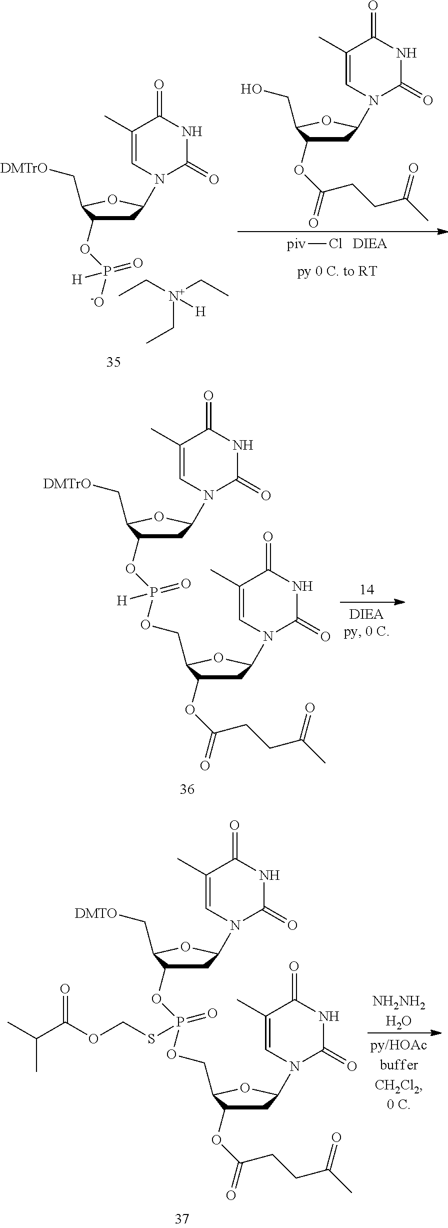

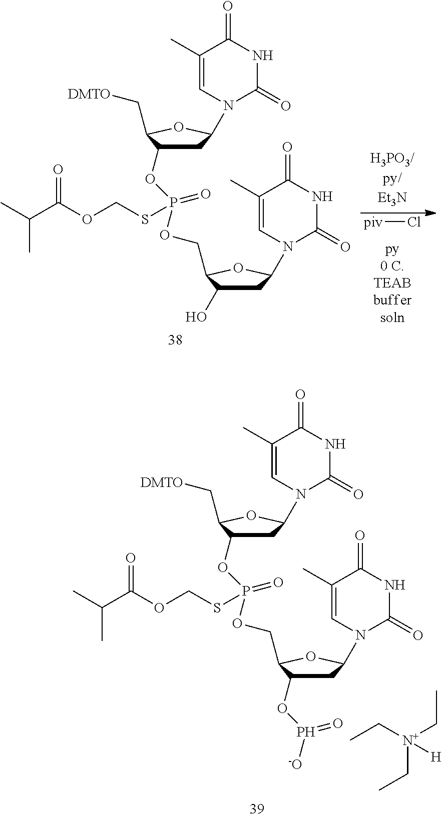

The H-phosphonate 35 (4.10 g, 5.77 mmol) and 5′-OH-T-Lev (1.71 g, 5.02 mmol) were placed in a one-neck round bottom flask and the mixture was coevaporated 3× on rotovap with pyridine (anhydrous 3×20 mL). The flask was then fitted with thermocouple, argon filled balloon, a stirbar, and a cooling ice-water bath. The oily residue was then dissolved in 20 mL of anhydrous pyridine. The reaction mixture was cooled to ice temperature at 0.5° C., and pivaloyl chloride (1.23 mL, 10.05 mmol) was added dropwise. The resulting solution was stirred for one hour at close to 0° C. The mixture was concentrated to near dryness on rotovap to remove acidic by-products. The residue was redissolved in 24 mL of anhydrous pyridine and the mixture containing the H-phosphonate 36 was then used for the sulfurization step. Anal. HPLC 58.9% purity.

To the cooled reaction mixture (containing 1.25 mmol) in preparation of 36, diluted with equal part anhydrous pyridine (6 mL) and cooled to 0° C. in ice bath, was added the resin-supported sulfurization reagent 14 (1.52 g, loading 1.15 mmol/g, 1.75 mmol, 1.4 equiv) and DIEA (393 μL, 2.26 mmol, 1.8 equiv). The cooling bath was removed and the mixture was stirred at ambient temperature for 60 mins. The reaction was monitored by HPLC. The mixture was filtered and the resin on the frit was washed extensively with dichloromethane (4×50 mL). The isolated resin was placed in vacuo overnight to give 1150 mg of used resin, and analyzed by elemental analysis and the ³¹P nmr test. The filtrate was quenched by adding to ice-cold saturated sodium bicarbonate solution (50 mL) and dichloromethane (50 mL). Extraction with dichloromethane (2×50 mL) followed by a rinse with brine (40 mL), drying over and filtering from MgSO₄, and finally concentration in vacuo provided crude 37 as a yellow brown oil. The product was purified and isolated using the standard protocol for precipitation from celite. Yield: 0.760 g (57%). Anal. ³¹P nmr shows diastereomers, 27.1 and 26.8 ppm; HPLC 85.9% purity.

To a round bottom flask purged with argon under a filled balloon was added the Lev-protected dimer 37 (1.71 g, 1.60 mmol) and coevaporation using anhydrous pyridine (2×15 mL) rendered the nucleotide anhydrous. Under argon atmosphere, added CH₂Cl₂ (4 mL, anhydrous). The mixture was stirred and cooled on ice bath, whereupon a cold solution of pyridine:acetic acid:hydrazine hydrate (3.0 mL:1.5 mL:0.11 mL) was then added via syringe; this is 1.4 equiv hydrazine. The buffered mixture was then stirred at 0° C. for 2 hrs. The mixture was added to cold CH₂Cl₂ and saturated sodium bicarbonate solution, extracted with CH₂Cl₂ (3×40 mL), rinsed quickly with brine, dried over MgSO₄. Filtration followed by concentration in vacuo provided crude 38, 1.94 g (>100%). This material was taken as is without purification for H-phosphonate formation. Anal. ³¹P nmr shows diastereomers, 27.8 and 26.8 ppm; HPLC 76.7% purity.

The 3′-OH dimer 38 (1.55 G, 1.60 mmol) was placed in a round bottom flask and then rendered anhydrous using 2 coevaporation cycles in anhydrous pyridine (2×20 mL). The residue was placed under argon atmosphere and 4 mL of anhydrous pyridine was added. To a separate flask, a previously prepared solution of phosphorous acid in pyridine-triethylamine mixture (4.49M, 4.65 mL, 20.91 mmol, 13 equiv.) was placed via a syringe. The flask was equipped with a thermocouple, magnetic stirrer, and balloon filled with argon. Anhydrous pyridine (5 mL) was added via syringe at ambient temperature, and the resulting solution was chilled on a water-ice bath to 4.5° C. Pivaloyl chloride (1.36 mL, 11.08 mmol, 6.9 equiv) was slowly added via syringe to the above cold solution of phosphorus acid with the exotherm taking the temperature to around 15° C. The resulting slurry was stirred for an additional 30 min, and the prepared nucleoside solution in pyridine was added dropwise to the H₃PO₃/pivaloyl chloride reagent. The cooling bath was removed, and the reaction was allowed to stir overnight at ambient temperature. The reaction was monitored by HPLC and TLC (5% MeOH in CH₂Cl₂) until starting material 38 was nearly was consumed (required about 21 hours). The reaction was subsequently quenched in a 500 mL Erlenmeyer flask equipped with magnetic stirrer, thermocouple, pH meter and containing a mixture of cold triethylammonium bicarbonate buffer (TEAB, 150 mL, pH 8.5) and dichloromethane (75 mL). A couple of chips of dry ice were added to bring the pH down from pH 9.1 to 8.5. The Erlenmeyer flask was also cooled in an ice bath. The reaction mixture was slowly transferred dropwise by pipet into the buffer with vigorous stirring. The quench was monitored so as to maintain temperature about 5-10° C. and the pH around 7-8. The pH dropped to a minimum of pH 7.3 and stabilized at ca. pH 7.6, while the temperature was 5.4° C. After completion of the quench, the aqueous phase was extracted with dichloromethane (3×75 mL, and the organic bottom layer was isolated and then washed with fresh TEAB buffer solution (ca. 60 mL). The extract was separated, dried over anhydrous magnesium sulfate, filtered, and concentrated in vacuo at ca. 35° C. to give >100% yield of 39 as a yellowish oil. The product was purified and isolated using the standard protocol for precipitation from celite. Yield: 1.44 g (79.5%). Anal. ³¹P nmr shows diastereomers, 26.7 and 26.6 ppm, 3.3 and 1.5 ppm; HPLC 91.2% purity

### Example 29

### Preparation of DMTrO-T(═S)T-OLev 40

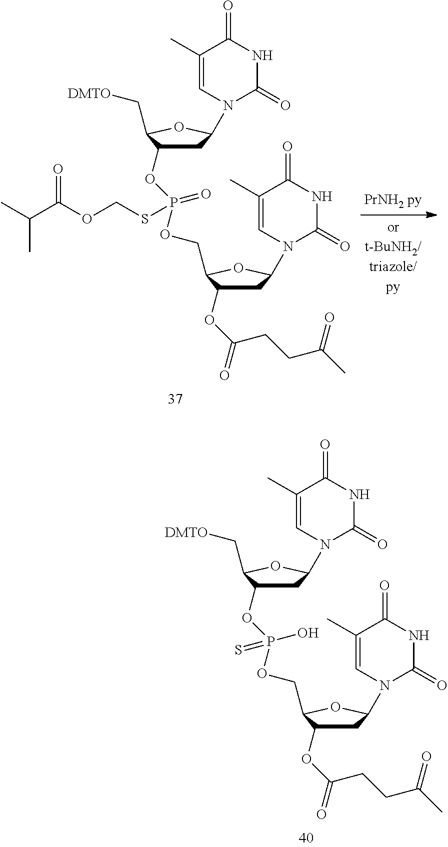

To a round bottom flask under argon purge was added the dimer 37 (434 mg, 0.408 mmol) and anhydrous pyridine (10 mL). The mixture was coevaporated to dryness twice to render the solution anhydrous. To the residue under argon was added molecular sieves, and anhydrous pyridine (4 mL). The mixture was stirred on an ice bath, and propylamine (670 μL, 20 equiv) was added by syringe. The mixture was stirred at room temperature for 3 hrs and then monitoring by ³¹P showed that at 19 hrs, the deprotection was complete. The solution was at this point stored in the freezer at −20° C. for future use. Anal. ³¹P nmr shows diastereomers, δ7.0 and 56.5 ppm, HPLC 78.0% purity

### Example 30

### Preparation of DMTrO-T(═S)T-OLev 40

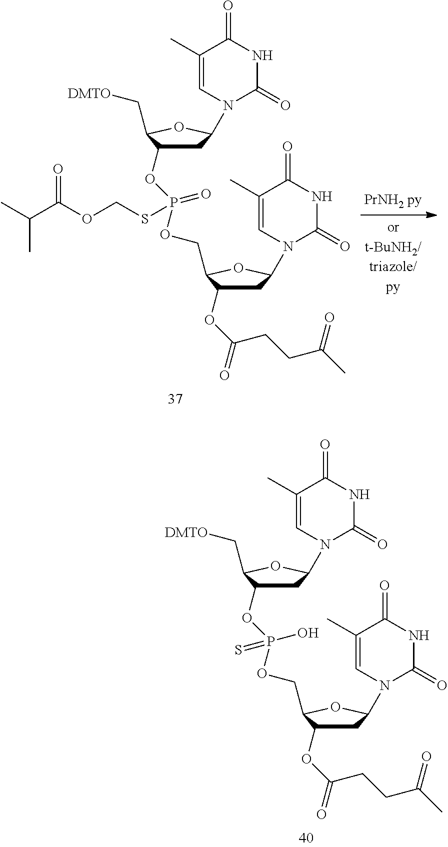

To a round bottom flask under argon purge was added the dimer 37 (246 mg, 0.231 mmol) and anhydrous pyridine (10 mL). The mixture was then coevaporated to dryness twice to render the solution anhydrous. To the residue under argon was added molecular sieves, 1,2,4-triazole (72 mg, 1.04 mmol, 4.5 equiv) and anhydrous pyridine (6 mL). The mixture was stirred on an ice bath, and tert-butylamine (733 μA, 6.94 mmol, 30 equiv) was added by syringe. The mixture was stirred at room temperature for 5 hrs. Monitoring by ³¹P showed that the deprotection was complete. The solution containing 40 was subsequently stored in the freezer at −20° C. for future use. Anal. ³¹P nmr shows diastereomers, δ7.9 and 57.5 ppm.

### Example 31

### Preparation of DMTrO-T(S—R^{B})T(H)T(S—R^{B})T-OLev 42

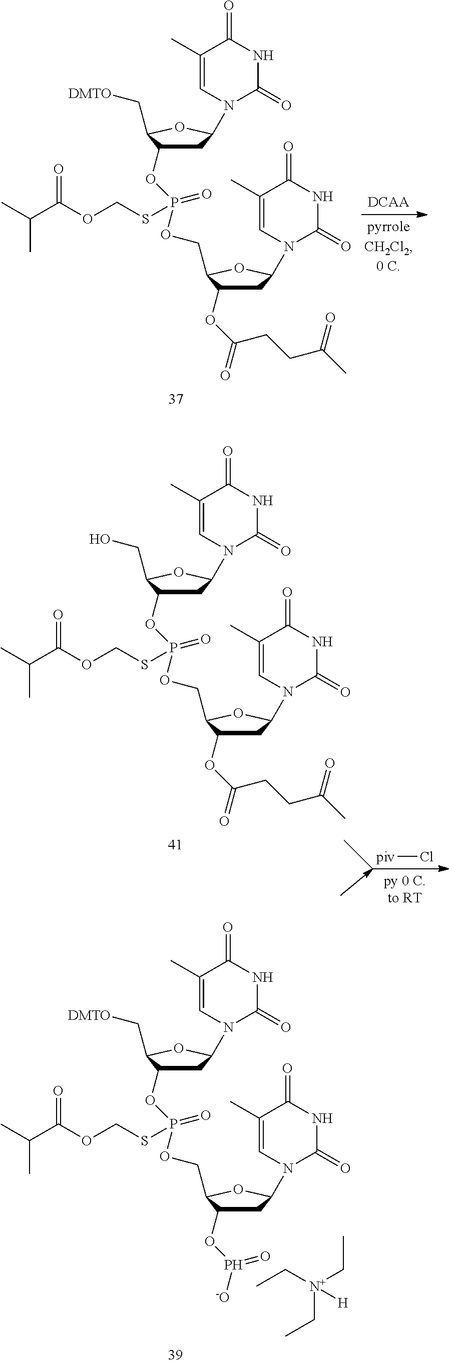

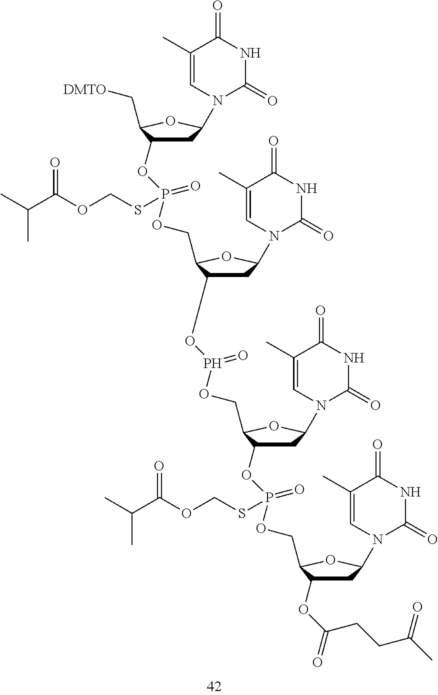

A mixture of dinucleotide 37 (1.11 g, 1.04 mmol) and pyrrole (442 μL, 6.37 mmol, 6.1 equiv) in 10 mL of dichloromethane was placed in a round bottom flask equipped with a magnetic stirrer, thermocouple, balloon filled with argon, and a cooling ice-methanol bath. The mixture was cooled to −11° C., and neat dichloroacetic acid (430 μL, 5.22 mmol, 5 equiv.) was added dropwise to the mixture. The resulting red solution stirred at this temperature for about 1 hour, and additional pyrrole (6 equiv) and DCAA (5 equiv) were added, maintaining the temperature close to 0° C. After 15 mins, the deep red-orange color disappeared and 37 also was consumed by TLC (41 was observed as a red spot with PMA, while 37 was observed as a bluish spot with PMA/heat, in 5% MeOH—CH₂Cl₂ eluent). The mixture was added to cold CH₂Cl₂ and saturated sodium bicarbonate solution, extracted with CH₂Cl₂ (3×60 mL), and dried over MgSO₄. Filtration followed by concentration in vacuo provided crude product. HO-T(S—R^{B})T-OLev, 41, was purified and isolated using the standard protocol for precipitation from celite. Yield: 0.694 g (87%). Anal. ³¹P nmr shows diastereomers, 27.4 and 27.3 ppm, HPLC 97.8% purity.

The H-phosphonate dimer 39 (1.18 g, 1.05 mmol) and HO-T(S—R^{B})T-OLev 41 (694 mg, 0.912 mmol) were placed in a one-neck round bottom flask and the mixture was rendered anhydrous by coevaporation 2× on rotovap with pyridine (anhydrous, 2×20 mL). The flask was next fitted with a thermocouple, argon filled balloon, a stirbar, and supported in a cooling ice-methanol bath. The oily residue was then dissolved in 10 mL of anhydrous pyridine. The reaction mixture was cooled to −14.1° C., and pivaloyl chloride (225 μL, 1.82 mmol) was added dropwise. The cooling bath was replaced with ice-water bath and the resulting solution was stirred for one hour while the internal temperature stabilized at −0.2° C. The mixture was then concentrated to near dryness on rotovap to remove acidic by-products. The residue was redissolved in 10 mL of anhydrous pyridine and the mixture then taken for sulfurization step. Anal. HPLC 90.6% purity.

### Example 32

### Preparation of DMTrO-T(═S)T(═S)T(═S)T-OLev 44

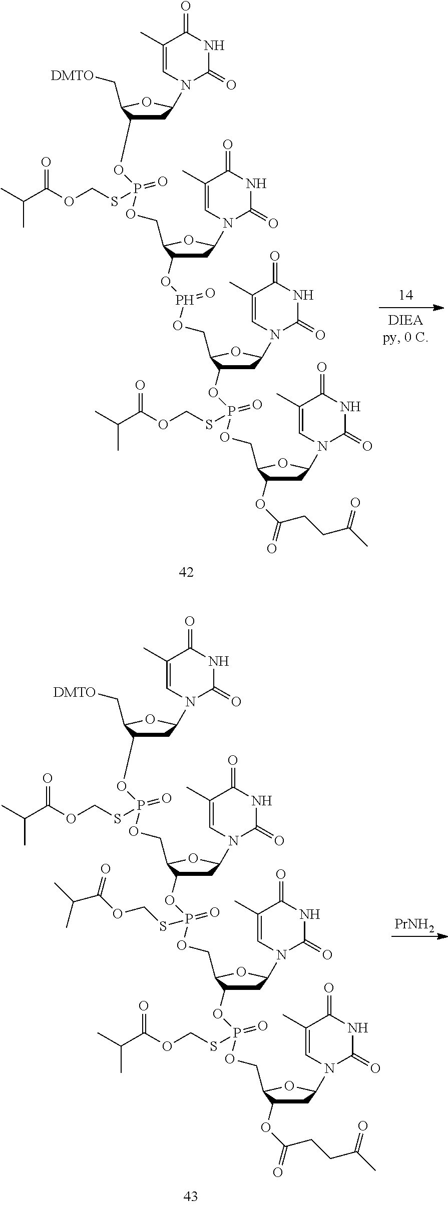

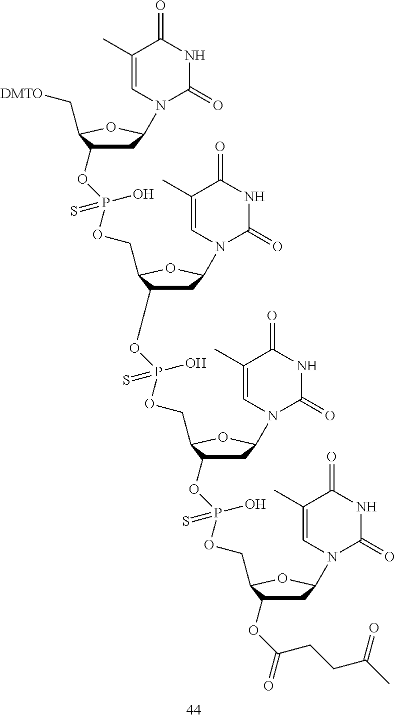

To the cooled reaction mixture (containing 0.547 mmol) in preparation of 42, cooled to 1.6° C. by thermocouple in an ice bath, was added under argon purge the resin-supported isobutyryl analog of the sulfurization transfer reagent 14 (856 mg, 0.984 mmol, 1.8 equiv) and DIEA (127 μL, 0.730 mmol, 2.0 equiv). The mixture was diluted with equal parts additional anhydrous pyridine (6 mL) and then stirred for 1.5 hrs. The reaction was monitored by HPLC. The mixture was filtered and the resin on the frit was washed extensively with dichloromethane (4×50 mL). The isolated resin was placed in vacuo overnight to give 708 mg of used resin, and analyzed by elemental analysis and the ³¹P nmr test. The filtrate was quenched by adding to ice-cold saturated sodium bicarbonate solution (100 mL) and dichloromethane (75 mL). Extraction with dichloromethane (3×75 mL) followed by a rinse with brine (60 mL), drying over and filtering from MgSO₄, and finally concentration in vacuo provided crude yellow brown oil. TLC (5% MeOH/DCM) showed product red spot at R_{f}=0.15 under PMA stain/heat and unreacted H-phosphonate at baseline. The crude product was loaded using dichloromethane onto a silica gel plug (3 cm×9 cm diameter) previously equilibrated in CH₂Cl₂. Elution first using dichloromethane, then 2% MeOH/DCM, followed by 5% MeOH/DCM and concentration of the fractions gave DMTrO-T(S—R^{B})T(S—R^{B})T(S—R^{B})T-OLev, 43, 786 mg (75%) as a foam. Anal. ³¹P nmr shows multiplet, 28.0 to 26.3 ppm; HPLC 93.2% purity.

To a round bottom flask under argon purge was added the tetramer 43 (259 mg, 0.136 mmol) and anhydrous pyridine. The solution was rendered anhydrous by coevaporation with anhydrous pyridine (2×10 mL). To the residue under argon was added molecular sieves, and anhydrous pyridine (3 mL). The mixture was stirred on an ice bath, and n-propylamine (671 uL, 8.16 mmol, 60 equiv) was added slowly. The mixture was then stirred at ambient temperature for 4 hrs. An aliquot (pyridine with D₂O insert) showed reaction was complete by ³¹P nmr. The solution containing tetramer 44 was then placed in the freezer at −20° C. for future use. Anal. ³¹P nmr shows multiplet, 57.9 to 55.4 ppm.

### Example 33

### Preparation of DMTrO-T(═S)T(═S)T(═S)T-OLev 44

To a round bottom flask under argon purge was added the tetramer 43 (177 mg, 0.093 mmol) and anhydrous pyridine. The solution was rendered anhydrous by coevaporation with anhydrous pyridine (2×10 mL). To the residue under argon was added molecular sieves, 1,2,4-triazole (86.7 mg, 13.5 equiv) and anhydrous pyridine (2 mL). The mixture was stirred on an ice bath, and tert-butylamine (883 uL, 90 equiv) was added. The mixture was then stirred at ambient temperature for 4 hrs. An aliquot (pyridine with D₂O insert) showed reaction was complete by ³¹P nmr. The solution containing tetramer 44 was then placed in the freezer at −20° C. for future use. Anal. ³¹P nmr shows multiplet, 56.9 to 55.2 ppm

### Example 34

### Preparation of DMTrO-T(═S)T(═S)T(═S)T-OLev 44

To a round bottom flask under argon purge was added the tetramer 43 (148 mg, 0.078 mmol) and anhydrous pyridine. The solution was rendered anhydrous by coevaporation with anhydrous pyridine (2×10 mL). To the residue under argon was added molecular sieves, and anhydrous pyridine (3 mL). The mixture was stirred on an ice bath, and methylamine (2.33 mL, 2.0M in THF, 60 equiv) was added. The mixture was subsequently stirred at ambient temperature for 4 hrs. An aliquot (pyridine with D₂O insert) showed reaction was complete by ³¹P nmr. The solution containing tetramer 44 was then placed in the freezer at −20° C. for future use. Anal. ³¹P nmr shows multiplet, 58.0 to 56.5 ppm.

### Example 35

### Isolation and Purification of Dimers, Tetramers, and Higher Oligonucleotides From Crude Mixture

The crude mixture is dissolved in a minimal amount of dichloromethane, typically in about 2-5 mL per 1 mmol of oligomer. An initial HPLC trace and ³¹P-NMR spectrum is obtained for the crude mixture. The dichloromethane solution is diluted with a mixture of MTBE and heptane by dropwise addition until the solution becomes cloudy. For lower oligomers, the MTBE:heptanes ratio is about 1:1. This ratio may be decreased for higher oligomers. About 1.5 g to 2 g of Celite per 1 mmol of oligomer is added to the mixture, and the mixture is stirred. Dropwise addition of the mixture of MTBE and heptanes is resumed until the volume of the second addition exceeds the volume of the first addition. The mixture is stirred for an additional 20 minutes. The suspension is filtered, and an HPLC trace for the filtrate is obtained. The Celite is washed with additional dichloromethane and the mixture of MTBE and heptanes. The Celite is finally washed extensively with additional dichloromethane to remove the product. The solvents are then removed in vacuo to yield the crude oligomer as a foam. An HPLC trace is obtained for this crude oligomer.

The crude sample is dissolved in about 5 ml to 8 ml of acetonitrile per 1 mmol of oligomer. The solution is cooled in an ice bath. With stirring, cold water is added dropwise until the solution becomes cloudy. The mixture is stirred for an additional 20 minutes. After stirring, the solution is allowed to settle, with the oligomer collecting as an oily layer on the bottom. The oligomer is separated, for example, by decantation, pipet, or separatory funnel. The oligomer is diluted with dichloromethane and extracted three times to isolate the product. The solution is washed with saturated sodium bicarbonate solution, dried over magnesium sulfate or sodium sulfate, and filtered. The solution was concentrated in vacuo to yield purified oligomer as a foam. The purity of the oligomer is determined by HPLC analysis and ³¹P-NMR.

The use of the terms “a” and “an” and “the” and similar references in the context of this disclosure (especially in the context of the following claims) are to be construed to cover both the singular and the plural, unless otherwise indicated herein or clearly contradicted by context. All methods and individual method steps described herein can be performed in any suitable order or simultaneously unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope, or range of equivalents, to which the appended claims are entitled. No language in the specification should be construed as indicating any non-claimed element as essential to the practice of the present disclosure.

All references, including printed publications, patent applications, and patents, cited herein are hereby incorporated by reference to the same extent as if each reference were individually and specifically indicated to be incorporated by reference and were set forth in its entirety herein.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represents further embodiments of the present disclosure and are included as a part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

1. A solid-supported sulfurization reagent for use in the synthesis of an oligonucleotide, the solid-supported sulfurization reagent comprising the following moeity: 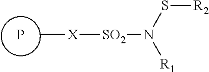
wherein (P) is the backbone of a polymeric solid support;
wherein X comprises a linker;
wherein R₁ comprises an alkyl group, a cycloalkyl group, an aryl group, or a heterocycle;
wherein R₂ comprises an alkyl group, an aryl group, a methyleneacyloxy group having the formula —CH₂—O—C(O)—R₇, a methylene carbonate group having the formula —CH₂—O—C(O)—OR₈, or a methylene carbamate group having the formula —CH₂—O—C(O)—NR₉R₁₀;
wherein R₇ comprises a saturated hydrocarbon residue having from 1 to 20 carbon atoms, an aromatic hydrocarbon residue having from 3 to 14 annular carbon atoms, or a heteroaromatic hydrocarbon residue having from 3 to 14 annular atoms, at least one of which is a nitrogen, oxygen, or sulfur atom;
wherein R₈ comprises a C₁ to C₂₀ alkyl group, cycloalkyl group, aryl group, or heteroaryl group;
wherein R₉ and R₁₀ independently comprises hydrogen, an alkyl group, a cycloalkyl group, and aryl group, or a heteroaryl group.

2. The solid-supported sulfurization reagent of claim 1, wherein the polymeric solid support comprises a polystyrene-based solid support.

3. The solid-supported sulfurization reagent of claim 1, wherein R₁ comprises an alkyl group or aryl group.

4. The solid-supported sulfurization reagent of claim 1, wherein the solid support comprises a polystyrene-based solid support, the linker comprises a phenyl group, R₁ is methyl, and R₂ is —CH₂OC(O)CH₂CH₃, —CH₂OC(O)CH(CH₃)₂, 4-chlorobenzene, 4-nitrobenzene, —CH₂OC(O)OCH₂CH₃, or —CH₂OC(O)N(CH₃)₂.

5. The solid-supported sulfurization reagent of claim 1, wherein the solid-supported sulfurization reagent comprises one of the following moieties: 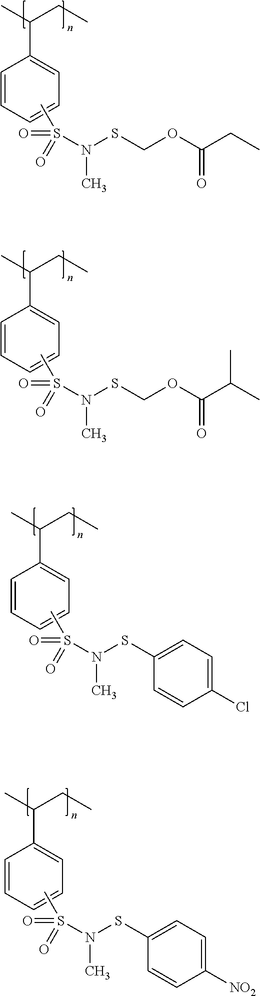
wherein n is 1 or higher, and wherein the moiety comprises an ortho-substituted, meta-substituted, or para-substituted phenyl group linker.

6. The solid-supported sulfurization reagent of claim 1, wherein the solid-supported sulfurization reagent has a load of sulfur transfer groups from about 0.1 mmol to about 4.5 mmol per 1 gram of the solid-supported sulfurization reagent.

7. The solid-supported sulfurization reagent of claim 1, wherein the solid-supported sulfurization reagent has a load of sulfur transfer groups from about 0.6 mmol to about 2.5 mmol per 1 gram of the solid-supported sulfurization reagent.

8. The solid-supported sulfurization reagent of claim 1, wherein the solid-supported sulfurization reagent has a load of sulfur transfer groups from about 1.0 mmol to about 1.5 mmol per 1 gram of the solid-supported sulfurization reagent.

9. A method of preparing a solid-supported sulfurization reagent comprising the steps of:
a. providing a solid-supported sulfonamide, wherein the solid-supported sulfonamide comprises the following moiety: 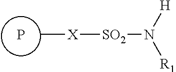
wherein (P) is the backbone of a polymeric solid support;
wherein X comprises a linker;
wherein R₁ comprises an alkyl group, a cycloalkyl group, an aryl group, or a heterocycle;
b. providing a sulfenyl chloride having the formula Cl—S—R₂,
wherein R₂ comprises an alkyl group, an aryl group, a methyleneacyloxy group having the formula —CH₂—O—C(O)—R₇, a methylene carbonate group having the formula —CH₂—O—C(O)—OR₈, or a methylene carbamate group having the formula —CH₂—O—C(O)—NR₉R₁₀;
wherein R₇ comprises a saturated hydrocarbon residue having from 1 to 20 carbon atoms, an aromatic hydrocarbon residue having from 3 to 14 annular carbon atoms, or a heteroaromatic hydrocarbon residue having from 3 to 14 annular atoms, at least one of which is a nitrogen, oxygen, or sulfur atom;
wherein R₈ comprises a C₁ to C₂₀ alkyl group, cycloalkyl group, aryl group, or heteroaryl group;
wherein R₉ and R₁₀ independently comprises hydrogen, an alkyl group, a cycloalkyl group, and aryl group, or a heteroaryl group; and
c. reacting the solid-supported sulfonamide with the sulfenyl chloride to yield the solid-supported sulfurization reagent comprising the following moiety:

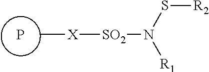

10. The method of claim 9, wherein the polymeric solid support comprises a polystyrene-based solid support.

11. The method of claim 9, wherein the solid support comprises a polystyrene-based solid support, the linker comprises a phenyl group, R₁ is methyl, and R₂ is selected from the group consisting of —CH₂OC(O)CH₂CH₃, —CH₂OC(O)CH(CH₃)₂, 4-chlorobenzene, 4-nitrobenzene, —CH₂OC(O)OCH₂CH₃, and —CH₂OC(O)N(CH₃)₂.

12. The method of claim 9, wherein the solid-supported sulfurization reagent comprises the one of the following moieties: 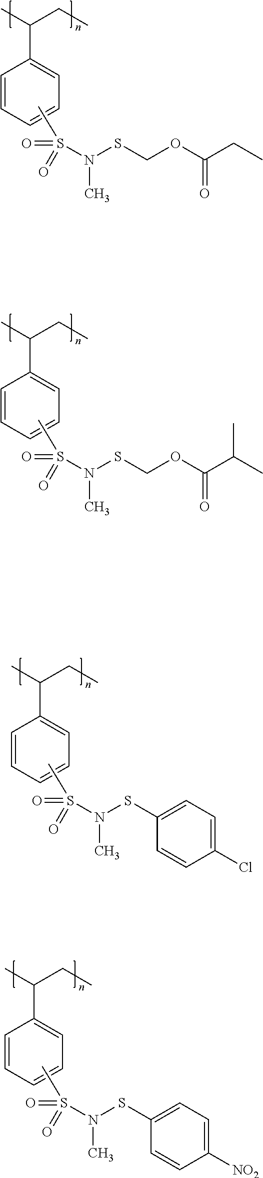
wherein n is 1 or higher, and wherein the moiety comprises an ortho-substituted, meta-substituted, or para-substituted phenyl group linker.

13. The method of claim 9, wherein the solid-supported sulfurization reagent has a load of sulfur transfer groups from about 0.1 mmol to about 4.5 mmol per 1 gram of the solid-supported sulfurization reagent.

14. The method of claim 9, wherein the solid-supported sulfurization reagent has a load of sulfur transfer groups from about 0.6 mmol to about 2.5 mmol per 1 gram of the solid-supported sulfurization reagent.

15. The method of claim 9, wherein the solid-supported sulfurization reagent has a load of sulfur transfer groups from about 1.0 mmol to about 1.5 mmol per 1 gram of the solid-supported sulfurization reagent.

16. A method of preparing a second oligonucleotide, wherein the second oligonucleotide comprises at least one P—S bond, the method comprising:
a. providing a first oligonucleotide containing at least one P—H bond;
b. providing a solid-supported sulfurization reagent, wherein the solid-supported sulfurization reagent comprises the following moiety: 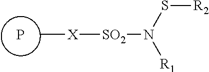
wherein (P) is the backbone of a polymeric solid support;
wherein X comprises a linker;
wherein R₁ comprises an alkyl group, a cycloalkyl group, an aryl group, or a heterocycle;
wherein R₂ comprises an alkyl group, an aryl group, a methyleneacyloxy group having the formula —CH₂—O—C(O)—R₇, a methylene carbonate group having the formula —CH₂—O—C(O)—OR₈, or a methylene carbamate group having the formula —CH₂—O—C(O)—NR₉R₁₀;
wherein R₇ comprises a saturated hydrocarbon residue having from 1 to 20 carbon atoms, an aromatic hydrocarbon residue having from 3 to 14 annular carbon atoms, or a heteroaromatic hydrocarbon residue having from 3 to 14 annular atoms, at least one of which is a nitrogen, oxygen, or sulfur atom;
wherein R₈ comprises a C₁ to C₂₀ alkyl group, cycloalkyl group, aryl group, or heteroaryl group;
wherein R₉ and R₁₀ independently comprises hydrogen, an alkyl group, a cycloalkyl group, and aryl group, or a heteroaryl group; and
c. reacting the first oligonucleotide with the solid-supported sulfurization reagent, wherein the N—SR₂ bond of the solid-supported sulfurization reagent is metathesized with the at least one P—H bond of the first oligonucleotide to yield the second oligonucleotide comprising at least one P—S bond and a solid-supported sulfonamide comprising an N—H bond.

17. The method of claim 16, wherein the solid-supported sulfurization reagent comprises one of the following moieties: 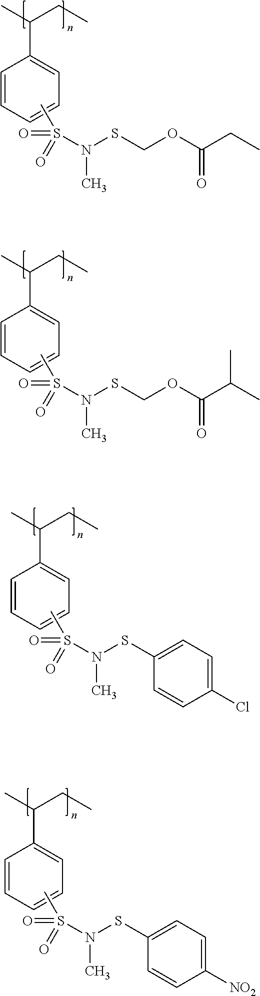
wherein n is 1 or higher, and wherein the moiety comprises an ortho-substituted, meta-substituted, or para-substituted phenyl group linker.

18. The method of claim 16, wherein the solid-supported sulfurization reagent has a load of sulfur transfer groups from about 0.1 mmol to about 4.5 mmol per 1 gram of the solid-supported sulfurization reagent.

19. The method of claim 16, wherein the solid-supported sulfurization reagent has a load of sulfur transfer groups from about 0.6 mmol to about 2.5 mmol per 1 gram of the solid-supported sulfurization reagent.

20. The method of claim 16, further comprising the steps of:
a. collecting the solid-supported sulfonamide by filtration; and
b. reacting the solid-supported sulfonamide with a sulfenyl chloride ClSR₂ to regenerate the solid-supported sulfurization reagent.
